(19) United States
(12) Patent Application Publication
(10) Pub. No.: US 2012/0115140 A1
(43) Pub. Date: May 10, 2012

(54) Molecular Diagnosis of Fragile X Syndrome Associated with FMR1 Gene

(75) Inventors: Scott A. Rivkees, Orange, CT (US); Jeffrey R. Gruen, Hamden, CT (US); Seiyu Hosono, Rye, NY (US); Karl Hager, Branford, CT (US)

(73) Assignees: JS GENETICS INC.; YALE UNIVERSITY

(21) Appl. No.: 13/266,434

(22) PCT Filed: Apr. 28, 2010
(86) PCT No.: PCT/US10/32797

§ 371 Date: Jan. 10, 2012

Related U.S. Application Data
(60) Provisional application No. 61/173,817, filed on Apr. 29, 2009.

(51) Int. Cl. C12Q 1/68

(52) U.S. Cl. 435/6.11; 435/6.12

(57) ABSTRACT

The present invention includes a rapid, selective, and accurate method of diagnosing a human subject with a triplet repeat genetic disorder of the FMR1 gene that leads to fragile X syndrome. The present invention also includes a rapid, selective, and accurate method of diagnosing a human subject at risk for developing a triplet repeat genetic disorder of the FMR1 gene that leads to fragile X syndrome, or at risk of passing such a disorder on to their progeny.

### BACKGROUND OF THE INVENTION

Triplet repeat genetic disorders, or trinucleotide repeat disorders, are human heritable disorders caused by trinucleotide repeats in certain genes that exceed a normal stable threshold. Trinucleotide repeat expansion, also known as triplet repeat expansion, is the DNA mutation responsible for causing any type of disorder categorized as a trinucleotide repeat disorder.

Triplet expansion is caused by slippage during DNA replication. Due to the repetitive nature of the DNA sequence in trinucleotide repeat regions, ‘loop out’ structures may form during DNA replication while maintaining complementary base paring between the parent strand and daughter strand being synthesized. If the loop out structure is formed from the sequence on the daughter strand, this results in an increase in the number of repeats. However, if the loop out structure is formed from the sequence on the parent strand, a decrease in the number of repeats occurs. Expansion of these repeats is more common than reduction. Generally, the larger the expansion the more likely that disease results and/or the severity of disease is increased. This property results in the characteristic of anticipation seen in trinucleotide repeat disorders. Anticipation describes the tendency of age of onset to decrease and severity of symptoms to increase through successive generations of an affected family due to expansion of these repeats.

As more repeat expansion diseases have been discovered, several categories have been established to group them based upon similar characteristics. Category I includes Huntington's disease (HD) and the spinocerebellar ataxias that are caused by a CAG repeat expansion in protein-coding portions of specific genes. Category H expansions tend to be more phenotypically diverse, including heterogeneous expansions that are generally small in magnitude, but which are more commonly found in gene exons. Category III includes fragile X syndrome, myotonic dystrophy, two of the spinocerebellar ataxias, juvenile myoclonic epilepsy, and Friedreich's ataxia. These diseases are characterized by much larger repeat expansions than is typically seen in either category I or II disorders, and the repeats are located outside of the protein-coding regions of the genes.

Triplet repeats are the site of mutation in each of these disorders. These repeats are GC-rich and highly polymorphic in the normal population. Fragile X syndrome is an example of a disease in which pre-mutation alleles cause little or no disease in the affected individual, but give rise to significantly amplified repeats in affected progeny.

Fragile X Syndrome (FRAX) is the most common genetic cause of mental retardation in males. The incidence of FRAX is about 1 per 4000 in males and 1 per 8000 in females. Females who have one abnormal Fragile-X and one normal-X chromosome may be normal or have mild manifestations of the FRAX syndrome. In addition FRAX may cause infertility in females.

Fragile X syndrome is caused by mutation of the FMR1 gene present on the X chromosome and occurs in 1 out of about every 2000 males and 1 out of about every 4000 females. Normally, the FMR1 gene contains between 4 and 45 repeats of the CGG trinucleotide sequence. There are four generally accepted forms of fragile X syndrome which relate to the length of the repeated CGG sequence; Normal (4-45 COO repeats), Premutation (60-200 CGG repeats), Full Mutation (more than 200 CGG repeats), and Intermediate or Gray Zone Alleles (45-60 repeats).

FRAX is caused by the expansion of a CGG trinucleotide repeat of the 5′ untranslated region (UTR) of the FMR1 gene located in chromosome band Xq27.3. In normal individuals, the 5′ UTR of the FMR1 gene contains 5 to 45 CGG repeats; however, individuals with FRAX have over 200 repeats. Expansion of the CGG repeats results in methylation of the promoter region, which silences the expression of the FMR1 protein (FMRP). FMRP normally binds to and facilitates the translation of a number of essential RNAs that are present in neurons. In FRAX, neuronal RNAs for FMR1 are not translated into protein leading to abnormal neural development via undefined mechanisms.

The FMR1 allele has over 200 CGG repeats in people with the fragile X syndrome. Expansion of the CGG repeats to such a degree results in methylation of that portion of the DNA, effectively silencing the expression of the FMR1 protein. The methylation of the FMR1 locus in chromosome band Xq27.3 is believed to result in constriction of the X chromosome which appears ‘fragile’ under the microscope at that point, a phenomenon that gave the syndrome its name.

Mutation of the FMR1 gene leads to the transcriptional silencing of the fragile X-mental retardation protein, FMRP. In normal individuals, FMRP binds and (usually) inhibits the translation of a number of essential neuronal RNAs. In fragile X patients, these RNAs are translated into excessive amounts of protein. However, certain RNAs seem to be stabilized by FMRP through a different mechanism.

In the prior art, fragile X syndrome is diagnosed by analysis of the number of CGG repeats and their methylation status using restriction endonuclease digestion and Southern blot analysis. This method is not suited to high-throughput screening, is labor intensive, and expensive. A disadvantage of Southern blotting is that this method requires large amounts of genomic DNA, and is slow and laborious. Thus, Southern blotting is not practical for population screening.

PCR protocols have been developed for assessing FMR1 CGG repeats with mixed success. Compared with Southern blot analysis, PCR testing is inexpensive, can be automated, and is fast. PCR can be performed on small amounts of DNA, making collection of samples convenient for patients. However, a major disadvantage of current PCR testing approaches for FRAX is that assay interpretation may not be straightforward or accurate for several reasons. First, PCR amplification of long CGG repeats is very difficult due to the highly GC-rich content of the region, especially in the presence of a second allele with fewer CGG repeats. Second, DNA fragments with expanded CGG repeats do not amplify well. This limitation is especially problematic for screening females and individuals with FRAX mosaicism, who have a normal FMR1 gene that will be preferentially amplified. To avoid these limitations, Southern blotting is performed on samples that fail to amplify by PCR and in females who appear to be homozygous normal.

Whereas quantitative DNA assays for the number and the methylation status of CGG repeats are available, there has been no quantitative assay for detecting the FMR1 protein (FMRP) levels using primary cells from patients. Until now, main approaches for measuring protein levels have been indirect and non-quantitative, involving immunohistochemical staining of blood smear or hair roots. Recently, an enzyme-linked immunosorbent assay (ELISA) for detecting FMR protein in peripheral blood lymphocytes was reported. However, this assay is not available for clinical use, and it is not clear that it can distinguish individual with and without FRAX.

A novel, rapid, accurate, and safe method for prenatal and postnatal screening and diagnosis of fragile X syndrome is urgently needed in the art. The present invention meets this need.

### SUMMARY OF THE INVENTION

One embodiment of the invention includes a method of diagnosing a human subject afflicted with fragile X syndrome, wherein fragile X syndrome is the result of an expansion of the CGG triplet repeat region of the FMR1 gene, the method comprising obtaining a sample of genomic DNA from the subject; contacting the sample with about 5-10 pairs of nested primers flanking the CGG triplet repeat region of the FMR1 gene; amplifying the CGG triplet repeat region of the FMR1 gene using Phi29 DNA polymerase for site specific multiple displacement amplification (SSMDA); and quantifying the number of CGG triplet repeats present in the CGG triplet repeat region of the FMR1 gene using either real-time PCR or real-time SSMDA, where if the number of CGG triplet repeats in the CGG triplet repeat region is more than about 200 CGG repeats, then the subject has fragile X syndrome. In one aspect, the sample of genomic DNA is contact with at least 2 primers selected from the group consisting of SEQ ID NO. 1-19.

Another embodiment of the invention includes a method of diagnosing a human subject with a fragile X syndrome premutation, wherein the fragile X syndrome is the result of an expansion of the CGG triplet repeat region of the FMR1 gene, wherein the subject is not afflicted with fragile X syndrome but is at-risk of having progeny with fragile X syndrome, the method comprising obtaining a sample of genomic DNA from the subject; contacting the sample with about 5-10 pairs of nested primers flanking the CGG triplet repeat region of the FMR1 gene; amplifying the CGG triplet repeat region of the FMR1 gene using Phi29 DNA polymerase for site specific multiple displacement amplification (SSMDA); and quantifying the number of CGG triplet repeats present in the CGG triplet repeat region of said FMR1 using either real-time PCR or real-time SSMDA, where if the number of CGG triplet repeats in the CGG triplet repeat region is from about 60 to about 200 CGG repeats, then the subject has a fragile X premutation and is at-risk of having progeny with fragile X syndrome.

Yet another embodiment of the invention includes a method of diagnosing a human subject with a fragile X syndrome intermediate premutation, wherein the fragile X syndrome intermediate premutation is the result of an expansion of the CGG triplet repeat region of the FMR1 gene, where the subject is not afflicted by fragile X syndrome but is at-risk of having progeny with fragile X syndrome, the method comprising obtaining a sample of genomic DNA from the subject; contacting the sample with about 5-10 pairs of nested primers flanking the CGG triplet repeat region of the FMR1 gene; amplifying the CGG triplet repeat region of said FMR1 gene using Phi29 DNA polymerase for site specific multiple displacement amplification (SSMDA); and quantifying the number of the CGG triplet repeats present in the CGG triplet repeat region of the FMR1 using either real-time PCR or real-time SSMDA, where if the number of CGG triplet repeats in the CGG triplet repeat region is from about 45 to about 60 CGG repeats, then the subject has an intermediate fragile X premutation and is at-risk of having progeny with fragile X syndrome.

Still another embodiment of the invention includes a method of diagnosing a human subject afflicted with fragile X syndrome, wherein said fragile X syndrome is the result of an expansion of the CGG triplet repeat region of the FMR1 gene, the method comprising obtaining a sample of genomic DNA from subject; digesting the genomic DNA with at least one restriction enzyme wherein the restriction enzyme excises a region of genomic DNA comprising the CGG triplet repeat region of the FMR1 gene; ligating the digested DNA to form circularized DNA comprising the CGG triplet repeat region of the FMR1 gene; contacting the circularized DNA with about 5-10 pairs of nested primers flanking the CGG triplet repeat region of the FMR1 gene; amplifying the CGG triplet repeat region of the FMR1 gene using Phi29 DNA polymerase for site specific multiple displacement amplification (SSMDA); and quantifying the number of CGG triplet repeats present in the CGG triplet repeat region of the FMR1 gene using either real-time PCR or real-time SSMDA, where if the number of CGG triplet repeats in the CGG triplet repeat region is more than about 200 CGG repeats, then the subject has fragile X syndrome.

Another embodiment of the invention includes a method of diagnosing a human subject with a fragile X premutation, wherein the fragile X premutation is the result of an expansion of the CGG triplet repeat region of the FMR1 gene, wherein the subject is not afflicted with fragile X syndrome but is at-risk of having progeny with fragile X syndrome, the method comprising obtaining a sample of genomic DNA from the subject; digesting the genomic DNA with at least one restriction enzyme wherein the restriction enzyme excises a region of genomic DNA comprising the CGG triplet repeat region of the FMR1 gene; ligating the digested DNA to form circularized DNA comprising the CGG triplet repeat region of the FMR1 gene; contacting the circularized DNA with about 5-10 pairs of nested primers flanking the CGG triplet repeat region of the FMR1 gene; amplifying the CGG triplet repeat region of the FMR1 gene using Phi29 DNA polymerase for site specific multiple displacement amplification (SSMDA); and quantifying the number of CGG triplet repeats present in the CGG triplet repeat region of the FMR1 gene using either real-time PCR or real-time SSMDA, where if the number of CGG triplet repeats in the CGG triplet repeat region is from about 60 to about 200 CGG repeats, then the subject has a fragile X premutation and is at-risk of having progeny with fragile X syndrome.

Still another embodiment of the invention includes a method of diagnosing a human subject with an intermediate fragile X premutation, wherein the fragile X intermediate premutation is the result of an expansion of the CGG triplet repeat region of the FMR1 gene, wherein the subject is not afflicted with fragile X syndrome but is at-risk of having progeny with fragile X syndrome, the method comprising obtaining a sample of genomic DNA from the subject; digesting the genomic DNA with at least one restriction enzyme wherein the restriction enzyme excises a region of genomic DNA comprising the CGG triplet repeat region of the FMR1 gene; ligating the digested DNA to form circularized DNA comprising the CGG triplet repeat region of the FMR1 gene; contacting the circularized DNA with about 5-10 pairs of nested primers flanking the CGG triplet repeat region of the FMR1 gene; amplifying the CGG triplet repeat region of the FMR1 gene using Phi29 DNA polymerase for site specific multiple displacement amplification (SSMDA); and quantifying the number of CGG triplet repeats present in the CGG triplet repeat region of the FMR1 gene using either real-time PCR or real-time SSMDA, where if the number of CGG triplet repeats in the CGG triplet repeat region is from about 45 to about 60 CGG repeats, then the subject has a fragile X intermediate premutation and is at-risk of having progeny with fragile X syndrome.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1, comprising FIG. 1A through FIG. 1C, depicts a representation of an approach to screen for FMR1:CGG triplet repeat. FIG. 1 is a schematic of the three step method of detecting CGG-repeat expansion in FRAX DNA. STEP 1: Whole Genome Amplification by Multiple Displacement Amplification (MDA) using a nucleotide analog 7-deaza-GTP. STEP 2: Enrichment of FMR1 CGG repeat region takes place using 7-deaza-GTP by Site-Specific Multiple Displacement Amplification (SSMDA) convert the genome into one with weaker G-C bonding with 7-deaza-Guanine instead of Guanine. STEP 3: TaqMan PCR to detect the CGG-repeat is performed with primer set F/R and a fluorescence reporter probe. FIG. 1B is a schematic illustration depicting the first mechanism of SSMDA reaction to amplify and enrich the 5′ untranslated region of the FMR1 gene for subsequent analysis. Eight pairs of flanking nested primers (1, 2, 3, 4, 5, 6, 7, 8) and 7-deaza-GTP are used to SSMDA amplify the CGG repeat region in the 5′UTR of FMR1 gene. FIG. 1C is a schematic illustration depicting the second mechanism of SSMDA reaction to amplify and enrich the 5′ untranslated region of the FMR1 gene for subsequent analysis.

FIG. 2, comprising FIG. 2A and FIG. 2B, is a series of images depicting the detection of CGG repeat copy number by quantitative real-time SSMDA and real-time PCR. FIG. 2A is a schematic diagram depicting the first mechanism to quantify the number of triplet codon repeats from the 5′ untranslated region of the fragile-X-associated FMR1 gene. FIG. 2B is a schematic diagram depicting the second mechanism to quantify the number of triplet codon repeats from the “amplified and enriched” 5′ untranslated region of fragile-X-associated FMR1 gene.

FIG. 3 depicts the amplification and enrichment of the FMR1 CGG repeat region by Sequence Specific Multiple Displacement Amplification (SSMDA). MDA primers are boxed. TaqMan primers are highlighted in grey. Control TaqMan primers are white letters on black background. Eight pairs of flanking nested primers (1, 2, 3, 4, 5, 6, 7, 8) are used to amplify the CGG repeat region in the 5′UTR of FMR1 gene (shown in box in the middle). F, Forward Primer; R, Reverse Primer. TaqMan primer sets: TaqMan Primer FXF and FXR.

FIG. 4, comprising FIGS. 4A and 4B demonstrates that the use of nucleotide analog 7-deaza-GTP in Whole Genome Amplification (WGA) with Multiple Displacement Amplification (MDA) and Site Specific Multiple Displacement Amplification (SSMDA) allows efficient amplification of CGG Repeats Region. FIG. 4A depicts dGTP was replaced by 7-deaza-GTP in both MDA whole genome amplification and SSMDA reaction. FIG. 4B depicts dGTP was used only in SSMDA reaction. X-axis shows the cycle number of the real-time PCR reaction. Y-axis shows the fluorescence intensity detected. Please note that using 7-deaza-GTP in both MDA and SSMDA allows us to distinguish CGG repeat size (FIG. 4A).

FIG. 5 is a schematic demonstrating the strategy of TaqMan Real-Time PCR used to detect the CGG triplet repeat expansion of the 5′UTR of the FMR1 gene. 5′FAM-CGCcGCCGCCGCCGC-MGB′3 (SEQ ID NO: 21) was used. F=FAM fluorophore. M=Minor Groove Binder Quencher.

FIG. 6 is a chart depicting differentiation of FRAX full mutation, premutation and non-FRAX (normal) genomic DNA using optimal PCR TaqMan condition. X-axis shows the cycle number of the TaqMan real-time PCR reaction, Y-axis, on the left, shows the fluorescence intensity (Fi) value detected. Y-axis, on the right, shows CGG repeat number. These data are representative of duplicate studies. Each line represents an individual sample. Please note the distinction between normal (<25), premutation (90-120) and full mutation (>200) CGG copy repeats. Please note sample designated “not known” is from a severely affected patient with FRAX, with unknown copy length of over 200 CGG repeats.

FIG. 7 is a chart depicting differences observed in the Ct Value (Ct) among samples with different CGG repeat. Graph depicts actual Ct values Ct (Y-axis) recorded in TaqMan assay. X-axis shows the different categories of FRAX samples tested based on the CGG repeat number. Duplicate experiments from 2 samples from female FRAX full mutation carrier, I female FRAX premutation carrier, 2 normal females, and 1 normal male did not show any significant difference in the Ct value. FRAX premutation and full mutation male samples were clearly distinguishable from each other based on the Ct value. The red bars connecting the categories show the corresponding p-values between the categories (p-value<0.05, ANOVA).

### DETAILED DESCRIPTION OF THE INVENTION

The present invention is based in part on the discovery of a rapid, selective, and accurate method of detecting triplet repeat genetic disorders in a human subject, including Fragile X Syndrome (FRAX). The present invention further includes a method of identifying a human subject carrying a premutation in the triplet repeat region of a gene that increases the likelihood that the progeny of that subject will be afflicted by a triplet repeat genetic disorder. The invention encompasses compositions, methods, and kits useful in detecting a triplet repeat mutation of the invention in a body sample obtained from a subject.

The invention provides a highly efficient and accurate screening assay for diagnosing FRAX. In one embodiment, the assay comprise three steps. In Step 1, Whole Genome Multiple Displacement Amplification is performed using, for example, 7-deaza-2-Guanosine (7-deaza GTP) nucleotide analog, which is incorporated into the whole genome. In Step 2, Site Specific Multiple Displacement Amplification (SSMDA) using, for example, 7-deaza GTP is performed to specifically enrich the CGG FMR1 expansion region and to weaken the GC base pairings, making the GCC expansion region more accessible to PCR (e.g., Taq DNA Polymerase in real-time PCT). In Step 3, SSMDA is followed by quantitative assessment of the numbers of CGG triplet repeats using PCR (e.g., TaqMan real-time PCR) without the need for sizing by gel electrophoresis or Southern blotting. Accordingly, the invention provides a means to clearly distinguish individuals with FRAX from unaffected individuals.

### DEFINITIONS

As used herein, each of the following terms has the meaning associated with it in this section.

The articles “a” and “an” are used herein to refer to one or to more than one (i.e. to at least one) of the grammatical object of the article. By way of example, “an element” means one element or more than one element.

The term “about” will be understood by persons of ordinary skill in the art and will vary to some extent on the context in which it is used.

“Amplified” DNA is DNA that has been “copied” once or multiple times, e.g. by polymerase chain reaction. When a large amount of DNA is available to assay, such that a sufficient number of copies of the locus of interest are already present in the sample to be assayed, it may not be necessary to “amplify” the DNA of the locus of interest into an even larger number of replicate copies. Rather, simply “copying” the template DNA once using a set of appropriate primers, which may contain hairpin structures that allow the restriction enzyme recognition sites to be double stranded, can suffice.

“Copy” as in “copied DNA” refers to DNA that has been copied once, or DNA that has been amplified into more than one copy.

By the term “applicator” as the term is used herein, is meant any device including, but not limited to, a hypodermic syringe, a pipette, a buccal swab, and other means for using the kits of the present invention.

As used herein, an “allele” is one of several alternate forms of a gene or non-coding regions of DNA that occupy the same position on a chromosome.

“Biological sample,” as that term is used herein, means a sample obtained from a subject, preferably a human, that can be used to as a source to obtain nucleic acid from that subject.

The phrase “body sample” as used herein, is intended any sample comprising a cell, a tissue, or a bodily fluid in which chromosomal material can be detected. Samples that are liquid in nature are referred to herein as “bodily fluids.” Body samples may be obtained from a patient by a variety of techniques including, for example, by scraping or swabbing an area or by using a needle to aspirate bodily fluids. In one embodiment, the body sample may be fluid obtained from a pregnant female, including saliva, urine, blood, or amniotic fluid. A body sample may also include cells or tissue obtained from a fetus. Biological samples include, without being limited to, amniotic fluid, chorionic villous biopsy, fetal cells in maternal circulation, fetal blood cells extracted from an umbilical artery or vein, fetal cells from premortem or postmortem tissues, and fixed tissue can be used in the methods of the present invention.

A “coding region” of a gene consists of the nucleotide residues of the coding strand of the gene and the nucleotides of the non-coding strand of the gene which are homologous with or complementary to, respectively, the coding region of an mRNA molecule which is produced by transcription of the gene.

“Complementary” as used herein refers to the broad concept of subunit sequence complementarity between two nucleic acids, e.g., two DNA molecules. When a nucleotide position in both of the molecules is occupied by nucleotides normally capable of base pairing with each other, then the nucleic acids are considered to be complementary to each other at this position. Thus, two nucleic acids are complementary to each other when corresponding positions in each of the molecules are occupied by nucleotides which normally base pair with each other (e.g., A:T and G:C nucleotide pairs).

“Substantially complementary to” refers to probe or primer sequences which hybridize to the sequences listed under stringent conditions and/or sequences having sufficient homology with test polynucleotide sequences, such that the allele specific oligonucleotide probe or primers hybridize to the test polynucleotide sequences to which they are complimentary.

The term “diagnose,” as used herein refers to a clinical practice of identifying a disease or condition in a subject by signs, symptoms, or results from an assay or test performed on the subject or a biological samples obtained from the subject.

The term “DNA” as used herein is defined as deoxyribonucleic acid.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

“Sequence variation” as used herein refers to any difference in nucleotide sequence between two different oligonucleotide or polynucleotide sequences.

“Polymorphism” as used herein refers to a sequence variation in a gene which is not necessarily associated with pathology.

“Mutation” as used herein refers to an altered genetic sequence which results in the gene coding for a non-functioning protein or a protein with substantially reduced or altered function. Generally, a deleterious mutation is associated with pathology or the potential for pathology.

As used herein, an “instructional material” includes a publication, a recording, a diagram, or any other medium of expression, which can be used to communicate the usefulness of the nucleic acid, peptide, and/or composition of the invention in the kit for effecting alleviation of the various diseases or disorders recited herein. Optionally, or alternately, the instructional material may describe one or more methods of alleviation the diseases or disorders in a cell or a tissue of a mammal. The instructional material of the kit of the invention may, for example, be affixed to a container, which contains the nucleic acid, peptide, chemical compound and/or composition of the invention or be shipped together with a container, which contains the nucleic acid, peptide, chemical composition, and/or composition. Alternatively, the instructional material may be shipped separately from the container with the intention that the instructional material and the compound be used cooperatively by the recipient.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids, which have been substantially purified from other components, which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA, which is part of a hybrid gene encoding additional polypeptide sequence.

Preferably, when the nucleic acid encoding the desired protein further comprises a promoter/regulatory sequence, the promoter/regulatory sequence is positioned at the 5′ end of the desired protein coding sequence such that it drives expression of the desired protein in a cell. Together, the nucleic acid encoding the desired protein and its promoter/regulatory sequence comprise a “transgene.”

In the context of the present invention, the following abbreviations for the commonly occurring nucleic acid bases are used. “A” refers to adenosine, “C” refers to cytidine, “G” refers to guanosine, “T” refers to thymidine, and “U” refers to uridine.

A “polynucleotide” means a single strand or parallel and anti-parallel strands of a nucleic acid. Thus, a polynucleotide may be either a single-stranded or a double-stranded nucleic acid.

A “portion” of a polynucleotide means at least about fifteen to about fifty sequential nucleotide residues of the polynucleotide. It is understood that a portion of a polynucleotide may include every nucleotide residue of the polynucleotide.

“Primer” refers to a polynucleotide that is capable of specifically hybridizing to a designated polynucleotide template and providing a point of initiation for synthesis of a complementary polynucleotide. Such synthesis occurs when the polynucleotide primer is placed under conditions in which synthesis is induced, i.e., in the presence of nucleotides, a complementary polynucleotide template, and an agent for polymerization such as DNA polymerase. A primer is typically single-stranded, but may be double-stranded. Primers are typically deoxyribonucleic acids, but a wide variety of synthetic and naturally occurring primers are useful for many applications. A primer is complementary to the template to which it is designed to hybridize to serve as a site for the initiation of synthesis, but need not reflect the exact sequence of the template. In such a case, specific hybridization of the primer to the template depends on the stringency of the hybridization conditions. Primers can be labeled with, e.g., chromogenic, radioactive, or fluorescent moieties and used as detectable moieties.

By the term “specifically binds,” as used herein, is meant a primer that recognizes and binds a complementary polynucleotide, but does not recognize and bind other polynucleotides in a sample.

### DESCRIPTION

The present invention encompasses a rapid, selective, and accurate method of diagnosing a human subject with a triplet repeat genetic disorder of the FMR1 gene that leads to fragile X syndrome. In one embodiment, the present invention includes a method of detecting an expansion of a triplet repeat region in the FMR1 gene in a human subject. If expansion of the triplet repeat region is detected in the gene, then the subject is identified or diagnosed as having either a premutation or a triplet repeat genetic disorder, depending on the magnitude of the triplet repeat expansion.

In another embodiment, the present invention includes a method of identifying a human subject at risk of having progeny afflicted with a triplet repeat genetic disorder of the FMR1 gene that leads to fragile X syndrome. Accordingly, the present invention includes a method of identifying a human subject with a premutation in the triplet repeat region of the FMR1 gene, wherein the premutation does not cause disease in the affected subject, but progeny of the affected subject have an increased likelihood of being afflicted with fragile X syndrome.

Accordingly, the invention encompasses compositions, methods, and kits useful in detecting an expansion of a triplet nucleotide repeat present in the FMR1 gene in a human subject.

### I. Compositions

The markers useful in the methods, assays, and kits of the present invention comprise, but are not limited to those listed in the table below. The Fragile-X FMR1 gene GCC triplet repeat expansion is depicted in SEQ ID NO: 1. Multiple Displacement Amplification (MDA) primers are depicted in Table 1. A phosphorothioate bond is present in the last two nucleotides of the 3′ end of the MDA primer. In quantitative, real-time PCR methods, a dual-labeled fluorogenic probe is used where a fluorescent reporter or fluorophore is conjugated to the 5′ end of the primer and a quencher is covalently attached to the 3′ end of the primer. A fluorescent reporter or fluorophore may include, but is not limited to, 6-carboxyfluorescein (FAM), tetrachlorofluorescein (TET), VIC (Applied Biosystems). A quencher includes tetramethylrhodamine (TAMRA) or dihydrocyclopyrroloindole tripeptide minor groove binder (MGB).

TABLE 1

| Name | | SEQ |
|---|---|---|
| F = forward | Primer sequence | ID |
| R = reverse | (5′ to 3′) | NO. |
| FRAX 1F | AACTGGGATAACCGGATGCA | 2 |
| FRAX 2F | AGTGCACCTCTGCAGAAAT | 3 |
| FRAX 3F | AGGCAGTGCGACCTGTCAC | 4 |
| FRAX 4F | TTCCCGCCCTCCACCAA | 5 |
| FRAX 5F | ACCCCGGCCGGTTCCCAGCA | 6 |
| FRAX 6F | AGGCCACTTGAAGAGAGA | 7 |
| FRAX 7F | AGCGTTGATCACGTGACGT | 8 |
| FRAX 8F | CAGCGGGCCGGGGGTTC | 9 |
| FRAX 1R | TCACTTAGCGCCGATTTC | 10 |
| FRAX 2R | CCCATCCCCAGCTCACCCC | 11 |
| FRAX 3R | ACCCTCTCCTCGCTGGTCT | 12 |
| FRAX 4R | GCCTCTCGGAGTCGGAGA | 13 |
| FRAX 5R | CAGTCCTTCCCTCCCAACAA | 14 |
| FRAX 6R | TGGCACCCAGGCGCGGT | 15 |
| FRAX 7R | CCTGCCTCCCGCCGACAC | 16 |
| FRAX 8R | GGAAGGAAGGGCGAAGAT | 17 |
| TaqMan FRAX F | GACGGAGGCGCCGCTGCCAGG | 18 |
| TaqMan FRAX R | TGGGCTGCGGGCGCTCGAGG | 19 |
| TaqMan FRAX Probe F | FAM-CGCCGCCGCCGCCGC-TAMRA | 20 |
| TaqMan FRAX Probe R | FAM-CGCCGCCGCCGCCGC-MGB | 21 |
| TaqMan CONTROL F | CAGCGGGCCGGGGGTTC | 22 |
| TaqMan CONTROL R | CCTGGCAGCGGCGCCTCCGT | 23 |
| TaqMan CONTROL | VIC-GAAGTGAAACCGAAACGGA- | 24 |
| Probe F | TAMRA | |
| TaqMan CONTROL | 5′VIC-GAAGTGAAACCGAAACGG | 25 |
| Probe R | A-MGB | |

### II. Methods

The present invention includes a method of diagnosing a human subject with fragile X syndrome that results from an expansion of the CGG triplet repeat region of the FMR1 gene. The method comprises isolating a sample of genomic DNA from a body sample obtained from a subject and contacting the sample with at least one pair of primers that flank the CGG triplet repeat region of the FMR1 gene. The phrase “nucleic acid sample” preferably refers to a sample of genomic DNA, but could also refer to a sample of other kinds of nucleic acids. The method further comprises elongating (amplifying) the CGG triplet repeat region of the FMR1 gene using Phi29 DNA polymerase for site specific multiple displacement amplification (SSMDA). Conditions suitable for elongation and amplification of a complementary nucleic acid using Phi29 DNA polymerase are described elsewhere herein. The method further comprises quantifying the number of CGG triplet repeats present in the CGG triplet repeat region of the FMR1 gene using either real-time PCR or SSMDA. If the number of CGG triplet repeats detected in the FMR1 gene is between about 4-45 CGG repeats, then the gene is identified as normal. If the number of CGG triplet repeats detected in the FMR1 gene is more than about 200 CGG repeats in both X-chromosome in female or single X-chromosome in male, then the gene is identified as having the full mutation and the subject is diagnosed as being afflicted with fragile X syndrome.

It will be obvious to the skilled artisan that the method of the invention may yield superior results if more than one pair of primers is used to amplify the DNA sample. Accordingly, the method of the present invention comprises contacting a nucleic acid sample obtained from the body sample of a subject with a set of primers comprising about 1-5, about 5-10, about 1-10, about 10-15, about 15-20, about 10-20, or about 1-20 pairs of nested primers, each of which is about 15-20 nucleotides long, and each of which flanks the loci of interest. Primer design is a skill well-within routine experimental design for the skilled artisan. In one embodiment, a primer of the invention is thiol-protected at the last two nucleotides of the 3′ end to protect the primer from endonucleoase activity. A set of nested primers of the invention should bind to regions of the template DNA about 20 base pairs apart provided that all primer pairs used to practice the method of the invention flank the CGG triplet repeat region of the FMR1 gene.

In another embodiment, the present invention includes a method of diagnosing a human subject with fragile X syndrome. The method comprises isolating a sample of genomic DNA from a body sample obtained from a subject and digesting the sample with at least one restriction enzyme with cleavage sites flanking the CGG triplet repeat region and the site specific multiple displacement amplification primer sets, resulting in a fragment of genomic DNA comprising the CGG triplet repeat region of the FMR1 gene. The resulting genomic DNA fragment is then ligated to form circularized DNA comprising the CGG triplet repeat region of the FMR1 gene. The circularize genomic DNA comprising the CGG triplet repeat region of the FMR1 gene is contacted with at least one pair of primers that flank the CGG triplet repeat region of the FMR1 gene. In one embodiment, the method of the present invention comprises contacting a nucleic acid sample obtained from the body sample of a subject with a set of primers comprising about 1-5, about 5-10, about 1-10, about 10-15, about 15-20, about 10-20, or about 1-20 pairs of nested primers, each of which is about 15-20 nucleotides long, and each of which flanks the loci of interest. The method further comprises elongating (amplifying) the CGG triplet repeat region of the FMR1 gene using Phi29 DNA polymerase for SSMDA. The number of CGG triplet repeats present in the CGG triplet repeat region of the genomic DNA is quantified using either real-time PCR or SSMDA. If the number of CGG triplet repeats detected in the FMR1 gene is between about 4-45 CGG repeats, then the gene is identified as normal. If the number of CGG triplet repeats detected in the FMR1 gene is more than about 200 CGG repeats in both X-chromosome in female or single X-chromosome in male, then the gene is identified as having the full mutation and the subject is diagnosed as being afflicted with fragile X syndrome.

The present invention further comprises a method of diagnosing a human subject with a fragile X syndrome premutation that results from an expansion of the CGG triplet region of the FMR1 gene and who is therefore at-risk of having progeny with fragile X syndrome. The method comprises isolating a nucleic acid sample from a body sample obtained from a subject and contacting the sample with at least one pair of primers that flank the CGG triplet repeat region of the FMR1 gene. In one embodiment, the method of the present invention comprises contacting a nucleic acid sample obtained from the body sample of a subject with a set of primers comprising about 1-5, about 5-10, about 1-10, about 10-15, about 15-20, about 10-20, or about 1-20 pairs of nested primers, each of which is about 15-20 nucleotides long, and each of which flanks the loci of interest. The method further comprises elongating (amplifying) the COO triplet repeat region of the FMR1 gene using Phi29 DNA polymerase for SSMDA. The number of CGG triplet repeats present in the CGG triplet repeat region of the genomic DNA is quantified using either real-time PCR or SSMDA. If the number of CGG triplet repeats detected in the FMR1 gene is between about 60-200 CGG repeats (i.e. the CGG triplet region is expanded) in one of the X-chromosome in female, then the gene is identified as having a “premutation” and the subject is at-risk of having progeny with fragile X syndrome.

In one embodiment, the present invention includes a method of diagnosing a human subject with fragile X syndrome premutation that results from an expansion of the CGG triplet repeat region of the FMR1 gene and who is therefore at-risk of having progeny with fragile X syndrome. The method comprises isolating a sample of genomic DNA from a body sample obtained from a subject and incubating the sample with at least one restriction enzyme with cleavage sites flanking the region of genomic DNA comprising the CGG triplet repeat region of the FMR1 gene and the site specific multiple displacement amplification primer sets. The resulting genomic DNA fragment is then ligated to form circularized genomic DNA comprising the CGG triplet repeat region of the FMR1 gene. The circularized genomic DNA comprising the CGG triplet repeat region of the FMR1 gene is contacted the sample with at least one pair of primers that flank the CGG triplet repeat region of the FMR1 gene. In one embodiment, the method of the present invention comprises contacting a nucleic acid sample obtained from the body sample of a subject with a set of primers comprising about 1-5, about 5-10, about 1-10, about 10-15, about 15-20, about 10-20, or about 1-20 pairs of nested primers, each of which is about 15-20 nucleotides long, and each of which flanks the loci of interest. The method further comprises elongating (amplifying) the CGG triplet repeat region of the FMR1 gene using Phi29 DNA polymerase for SSMDA. The number of CGG triplet repeats present in the CGG triplet repeat region of the genomic DNA is quantified using either real-time PCR or SSMDA. If the number of CGG triplet repeats detected in the FMR1 gene is between about 55-200 CGG repeats (i.e. the CGG triplet region is expanded), then the gene is identified as having a “premutation” and the subject is at-risk of having progeny with fragile X syndrome.

In another embodiment, the present invention includes a method of diagnosing a human subject with fragile X syndrome “intermediate” premutation that results from an expansion of the CGG triplet repeat region of the FMR1 gene and who is therefore at-risk of having progeny with fragile X syndrome. In one embodiment, the method of the present invention comprises contacting a nucleic acid sample obtained from the body sample of a subject with a set of primers comprising about 1-5, about 5-10, about 1-10, about 10-15, about 15-20, about 10-20, or about 1-20 pairs of nested primers, each of which is about 15-20 nucleotides long, and each of which flanks the loci of interest. The method further comprises elongating (amplifying) the CGG triplet repeat region of the FMR1 gene using Phi29 DNA polymerase for SSMDA. If the number of CGG repeats detected in the FMR1 gene is between about 45-60 CGG repeats, then the gene is identified as having an “intermediate” premutation and the subject may be at risk of having progeny with fragile X syndrome.

In another embodiment, the present invention includes a method of diagnosing a human subject with fragile X syndrome “intermediate” premutation that results from an expansion of the COO triplet repeat region of the FMR1 gene and who is therefore at-risk of having progeny with fragile X syndrome. In still another embodiment, the method comprises isolating a sample of genomic DNA from a body sample obtained from a subject and incubating the sample with at least one restriction enzyme with cleavage sites flanking the region of genomic DNA comprising the CGG triplet repeat region of the FMR1 gene and the site specific multiple displacement amplification primer sets. The resulting genomic DNA fragment is then ligated to form circularized genomic DNA comprising the CGG triplet repeat region of the FMR1 gene. The circularize genomic DNA comprising the CGG triplet repeat region of the FMR1 gene is contacted the sample with at least one pair of primers that flank the CGG triplet repeat region of the FMR1 gene. In one embodiment, the method of the present invention comprises contacting a nucleic acid sample obtained from the body sample of a subject with a set of primers comprising about 1-5, about 5-10, about 1-10, about 10-15, about 15-20, about 10-20, or about 1-20 pairs of nested primers, each of which is about 15-20 nucleotides long, and each of which flanks the loci of interest. The method further comprises elongating (amplifying) the CGG triplet repeat region of the FMR1 gene using Phi29 DNA polymerase for SSMDA. The number of CGG triplet repeats present in the CGG triplet repeat region of the genomic DNA is quantified using either real-time PCR or SSMDA. If the number of CGG repeats detected in the FMR1 gene is between about 45-60 CGG repeats, then the gene is identified as having an “Intermediate” premutation and the subject may be at risk of having progeny with fragile X syndrome.

The method may be practiced on a subject, preferably a mammal, more preferably a human. In one embodiment, the subject is a pregnant woman. A body sample of the invention may be obtained from a subject at an appropriate period of pregnancy. Preferably, the body sample is obtained from a subject during the first or second trimesters of pregnancy. In another embodiment, the subject is a fetus, a neonate, or a child. In another embodiment, a subject of the invention is any human subject undergoing genetic counseling. Methods of collecting biological samples from a mother and/or fetus are well known in the art and include amniocentesis, venous blood draw, and standard histology or pathology techniques.

A loci of interest to be amplified may be selected based on sequence alone. In one embodiment, a triplet repeat region of a gene comprises a loci of interest. In another preferred embodiment, the CGG triplet repeat region of the FMR1 gene comprises a loci of interest. Any method of amplifying a loci of interest of a gene may be used in the practice of the invention for the purpose of enriching the nucleic acid sample for the loci of interest. However, a preferred method is site specific multiple displacement amplification (SSMDA) and real-time PCR. It will be appreciated by the skilled artisan that is may be desirable to digest a nucleic acid sample with at least one restriction enzyme that has digest sites that flank the loci of interest as described elsewhere herein.

In another embodiment, the method of the invention comprises contacting the nucleic acid sample obtained from the body of a subject with at least one six-cutter restriction enzyme that flanks the region of the gene where the set of nested primers will anneal. The digested DNA is diluted to about 0.1 to 10 nanogram per microlitre and ligated with DNA ligase to facilitate a monomolecular ligation event resulting in circular DNA comprising the target sequence. The resulting circular DNA is amplified using the primers and methods described above.

In all embodiments, quantification of the isolated DNA enriched for the 5′ region of the FMR1 gene is accomplished using either real-time SSMDA or real-time PCR as detailed elsewhere herein.

### A. Nucleic Acid Template

The method of the invention includes isolating a nucleic acid sample from a body sample obtained from a subject, enriching the sample for a loci of interest using methods of amplification known in the art, and screening the nucleic acid sample for expansion of at least one triplet repeat sequence associated with a triplet repeat genetic disorder. In a preferred embodiment, the method of the invention uses Sequence Specific Multiple Displacement Amplification (SSMDA) followed by quantification of the number of triplet repeats by real-time polymerase chain reaction (RT-PCR) of the SSMDA enriched region of the gene of interest. If expansion of at least one triplet repeat sequence is detected, then the subject is identified as having either a premutation or a triplet repeat genetic disorder, depending on the magnitude of the triplet repeat expansion.

In one embodiment, the method of the invention includes isolating a nucleic acid sample from a body sample obtained from a subject and screening the nucleic acid sample for expansion of at least one triplet repeat sequence associated with a triplet repeat genetic disorder using SSMDA followed by quantification of the number of triplet repeats by Real Time PCR of the SSMDA enriched region of the gene of interest. If a pre-mutation expansion of at least one triplet repeat sequence is detected, then the subject is identified as having a triplet repeat genetic pre-mutation and is at-risk of having progeny with a triplet repeat genetic disorder.

A nucleic acid sample is any type of nucleic acid sample in which potential triplet repeats in a gene, including the FMR1 gene, exist. For instance, the nucleic acid sample may be an isolated genome or a portion of an isolated genome. An isolated genome consists of all of the DNA material from a particular organism, i.e., the entire genome. A portion of an isolated genome, which is referred to as a reduced complexity genome (RCG), is a plurality of DNA fragments within an isolated genome but which does not include the entire genome. Genomic DNA comprises the entire genetic component of a species excluding, when applicable, mitochondrial DNA.

In one embodiment, the nucleic acid is amplified directly in the original sample containing the source of nucleic acid. It is not essential that the nucleic acid be extracted, purified or isolated; it only needs to be provided in a form that is capable of being amplified. Hybridization of the nucleic acid template with primer, prior to amplification, is not required. For example, amplification can be performed in a cell or sample lysate using standard protocols well known in the art. DNA that is on a solid support, in a fixed biological preparation, or otherwise in a composition that contains non-DNA substances and that can be amplified without first being extracted from the solid support or fixed preparation or non-DNA substances in the composition can be used directly, without further purification, as long as the DNA can anneal with appropriate primers, and be copied, especially amplified, and the copied or amplified products can be recovered and utilized as described herein.

In another embodiment, the nucleic acid is extracted, purified or isolated from non-nucleic acid materials that are in the original sample using methods known in the art prior to amplification.

In another embodiment, the nucleic acid is extracted, purified or isolated from the original sample containing the source of nucleic acid and prior to amplification, the nucleic acid is fragmented using any number of methods well known in the art including but not limited to enzymatic digestion, manual shearing, or sonication. For example, the DNA can be digested with one or more restriction enzymes that have a recognition site, and especially an eight base or six base pair recognition site, which is not present in the loci of interest, but rather flanks the loci of interest.

Fragments of DNA that contain the loci of interest can be purified from the fragmented DNA before amplification. Such fragments can be purified by using primers that will be used in the amplification (see “Primer Design” section below) as hooks to retrieve the loci of interest, based on the ability of such primers to anneal to the loci of interest. In a preferred embodiment, tag-modified primers are used, such as e.g. biotinylated primers.

By purifying the DNA fragments containing the loci of interest, the specificity of the amplification reaction can be improved. This will minimize amplification of nonspecific regions of the template DNA.

### B. Primer Design

The primers can be random, specific, a combination thereof, or hybrids containing a unique portion and a random portion. When the primer is random, the primer is preferably from about 6 to about 30 nucleotides in length. When the primer is specific, the primer is preferably from about 12 to about 50 nucleotides in length and can include some degenerate bases.

Published sequences, including consensus sequences, can be used to design or select primers for use in amplification of template DNA. The selection of sequences to be used for the construction of primers that flank a locus of interest can be made by examination of the sequence of the loci of interest, or immediately thereto. The recently published sequence of the human genome provides a source of useful consensus sequence information from which to design primers to flank a desired human gene locus of interest.

In a preferred embodiment, specific primers derived from unique sequences flanking the CGG triplet repeat region of the FMR1 gene are used to enrich the CGG triplet repeat region.

By “flanking” a locus of interest is meant that the sequences of the primers are such that at least a portion of the 3′ region of one primer is complementary to the antisense strand of the template DNA and upstream from the locus of interest site (forward primer), and at least a portion of the 3′ region of the other primer is complementary to the sense strand of the template DNA and downstream of the locus of interest (reverse primer). A “primer pair” is intended a pair of forward and reverse primers. Both primers of a primer pair anneal in a manner that allows extension of the primers, such that the extension results in amplifying the template DNA in the region of the locus of interest.

Examples of such primers include random or partially random primers depicted in Table 1.

It is preferred that, when amplifying minute amounts of DNA, the total amount of primers added to the reaction mix is between 1 ng and 10 μg. It is more preferred that the amount of primers is between 5 ng and 5 μg. It is most preferred that the amount of primers is between 25 ng and 2 μg.

The primers can contain any number of modifications including, but not limited to, modified bases, such as thiol protected nucleotide analogs, phosphorothioate bases, deoxyinosine, and 5-nitroindole, and the incorporation of detectable labels, such as biotin, fluorescein, and other dyes.

Primers can be prepared by a variety of methods including but not limited to cloning of appropriate sequences and direct chemical synthesis using methods well known in the art (Narang et al., 1979, Methods Enzymol. 68:90; Brown et al., 1979, Methods Enzymol. 68:109). Primers can also be obtained from commercial sources such as Operon Technologies, Amersham Pharmacia Biotech, Sigma, and Life Technologies, IDT Technologies. The primers can have an identical melting temperature. The lengths of the primers can be extended or shortened at the 5′ end or the 3′ end to produce primers with desired melting temperatures. In a preferred embodiment, one of the primers of the prime pair is longer than the other primer. In a preferred embodiment, the 3′ annealing lengths of the primers, within a primer pair, differ. Also, the annealing position of each primer pair can be designed such that the sequence and length of the primer pairs yield the desired melting temperature. The simplest equation for determining the melting temperature of primers smaller than 25 base pairs is the Wallace Rule (Td=2(A+T)+4(G+C)). Computer programs can also be used to design primers, including but not limited to Array Designer Software (Arrayit Inc.), Oligonucleotide Probe Sequence Design Software for Genetic Analysis (Olympus Optical Co.), NetPrimer, and DNAs is from Hitachi Software Engineering. The TM (melting or annealing temperature) of each primer is calculated using software programs such as Net Primer (free web based program at http://premierbiosoft.com/netprimer/netprlaunch/netprlaunch.html; internet address as of Apr. 17, 2002).

In another embodiment, the annealing temperature of the primers can be recalculated and increased after any cycle of amplification, including but not limited to cycle 1, 2, 3, 4, 5, cycles 6-10, cycles 10-15, cycles 15-20, cycles 20-25, cycles 25-30, cycles 30-35, or cycles 35-40. After the initial cycles of amplification, the 5′ half of the primers is incorporated into the products from each loci of interest, thus the TM can be recalculated based on both the sequences of the 5′ half and the 3′ half of each primer.

As used herein, the term “about” with regard to annealing temperatures is used to encompass temperatures within 10 degrees Celsius (° C.) of the stated temperatures.

### C. Detection Labels

To aid in detection and quantitation of nucleic acids amplified using the disclosed method, detection labels can be directly incorporated into amplified nucleic acids or can be coupled to detection molecules. As used herein, a detection label is any molecule that can be associated with amplified nucleic acid, directly or indirectly, and which results in a measurable, detectable signal, either directly or indirectly. Many such labels for incorporation into nucleic acids or coupling to nucleic acid probes are known to those of skill in the art. Examples of detection labels suitable for use in the disclosed method are radioactive isotopes, fluorescent molecules, phosphorescent molecules, enzymes, antibodies, and ligands.

Examples of suitable fluorescent labels include fluorescein (FITC), 5,6-carboxymethyl fluorescein, Texas red, nitrobenz-2-oxa-1,3-diazol-4-yl (NBD), coumarin, dansyl chloride, rhodamine, 4′-6-diamidino-2-phenylinodole (DAPI), and the cyanine dyes Cy3, Cy3.5, Cy5, Cy5.5 and Cy7. Preferred fluorescent labels are fluorescein (5-carboxyfluorescein-N-hydroxysuccinimide ester) and rhodamine (5,6-tetramethyl rhodamine). Preferred fluorescent labels are FITC and the cyanine dyes Cy3, Cy3.5, Cy5, Cy5.5 and Cy7. The absorption and emission maxima, respectively, for these fluors are: FITC (490 nm; 520 nm), Cy3 (554 nm; 568 nm), Cy3.5 (581 nm; 588 nm), Cy5 (652 nm: 672 nm), Cy5.5 (682 nm; 703 nm) and Cy7 (755 nm; 778 nm), thus allowing their simultaneous detection. The fluorescent labels can be obtained from a variety of commercial sources, including Molecular Probes, Eugene, Oreg. and Research Organics, Cleveland, Ohio.

Labeled nucleotides are a preferred form of detection label since they can be directly incorporated into the amplification products during synthesis. Examples of detection labels that can be incorporated into amplified DNA or RNA include nucleotide analogs such as BrdUrd (Hoy and Schimke, 1993, Mutation Research 290:217-230), BrUTP (Wansick et al., 1993, J. Cell Biology 122:283-293) and nucleotides modified with biotin (Langer et al., 1981, Proc. Natl. Acad. Sci. USA 78:6633) or with suitable haptens such as digoxygenin (Kerkhof, 1992, Anal, Biochem. 205:359-364). Suitable fluorescence-labeled nucleotides are Fluorescein-isothiocyanate-dUTP, Cyanine-3-dUTP and Cyanine-5-dUTP (Yu et al., 1994, Nucleic Acids Res., 22:3226-3232). A preferred nucleotide analog detection label for DNA is BrdUrd (BUDR triphosphate, Sigma), and a preferred nucleotide analog detection label for RNA is Biotin-16-uridine-5′-triphosphate (Biotin-16-dUTP, Boehringher Mannheim). Fluorescein, Cy3, and Cy5 can be linked to dUTP for direct labeling. Cy3.5 and Cy7 are available as avidin or anti-digoxygenin conjugates for secondary detection of biotin- or digoxygenin-labeled probes.

Detection labels that are incorporated into amplified nucleic acid, such as biotin, can be subsequently detected using sensitive methods well-known in the art. For example, biotin can be detected using streptavidin-alkaline phosphatase conjugate (Tropix, Inc.), which is bound to the biotin and subsequently detected by chemiluminescence of suitable substrates (for example, chemiluminescent substrate CSPD: disodium, 3-(4-methoxyspiro-[1,2,dioxetane-3-2′-(5′-chloro)tricyclo[3.3.1.1.^{3,7}]decane]-4-yl) phenyl phosphate; Tropix, Inc.).

A preferred fluorescent probe used in the methods of the invention includes 5′FAM-(GCC)n-MGB′3 to detect the CGG expansion. A preferred fluorescent probe used in the methods of the invention includes 5′VIC-(FMR1 gene)-MGB′3 to detect an internal control sequence, which serves as an internal reference for detecting the CGG repeat copy number.

Molecules that combine two or more of these detection labels are also considered detection labels. Any of the known detection labels can be used with the disclosed probes, tags, and method to label and detect nucleic acid amplified using the disclosed method. Methods for detecting and measuring signals generated by detection labels are also known to those of skill in the art. For example, radioactive isotopes can be detected by scintillation counting or direct visualization; fluorescent molecules can be detected with fluorescent spectrophotometers; phosphorescent molecules can be detected with a spectrophotometer or directly visualized with a camera; enzymes can be detected by detection or visualization of the product of a reaction catalyzed by the enzyme; antibodies can be detected by detecting a secondary detection label coupled to the antibody. As used herein, detection molecules are molecules which interact with amplified nucleic acid and to which one or more detection labels are coupled.

### D. Strand Displacement Amplification

### 1. DNA Polymerases

DNA polymerases useful in the multiple displacement amplification must be capable of displacing, either alone or in combination with a compatible strand displacement factor, a hybridized strand encountered during replication. Such polymerases are referred to herein as strand displacement DNA polymerases. It is preferred that a strand displacement DNA polymerase lack a 5′ to 3′ exonuclease activity. Strand displacement is necessary to result in synthesis of multiple copies of a target sequence. A 5′ to 3′ exonuclease activity, if present, might result in the destruction of a synthesized strand. It is also preferred that DNA polymerases for use in the disclosed method are highly processive. The suitability of a DNA polymerase for use in the disclosed method can be readily determined by assessing its ability to carry out strand displacement replication. Preferred strand displacement DNA polymerases include, but are not limited to bacteriophage Phi29 DNA polymerase (U.S. Pat. Nos. 5,198,543 and 5,001,050 to Blanco et al.), Bst large fragment DNA polymerase (Exo(−) Bst; Aliotta et al., 1996, Genet. Anal. (Netherlands) 12:185-195) and exo(−)Bca DNA polymerase (Walker and Linn, 1996, Clinical Chemistry 42:1604-1608). Other useful polymerases include, but are not limited to, phage M2 DNA polymerase (Matsumoto et al., 1989, Gene 84:247), phage φPRD1 DNA polymerase (Jung et al., 1987, Proc. Natl. Acad. Sci. USA 84:8287), exo(−) VENT® DNA polymerase (Kong et al., 1993, J. Biol. Chem. 268:1965-1975), Klenow fragment of DNA polymerase I (Jacobsen et al., 1974, Eur. J. Biochem. 45:623-627), T5 DNA polymerase (Chatterjee et al., 1991, Gene 97:13-19), Sequenase (U.S. Biochemicals), PRD1 DNA polymerase (Zhu and Ito, 1994, Biochim. Biophys. Acta. 1219:267-276), and T4 DNA polymerase holoenzyme (Kaboord and Benkovic, 1995, Curr. Biol. 5:149-157).

Strand displacement can be facilitated through the use of a strand displacement factor, such as helicase. It is considered that any DNA polymerase that can perform strand displacement replication in the presence of a strand displacement factor is suitable for use in the disclosed method, even if the DNA polymerase does not perform strand displacement replication in the absence of such a factor. Strand displacement factors useful in strand displacement replication include BMRF1 polymerase accessory subunit (Tsurumi et al., 1993, J. Virology 67(12)3648-7653), adenovirus DNA-binding protein (Zijderveld and van der Vliet, 1994, J. Virology 68(2):1158-1164), herpes simplex viral protein ICP8 (Boehmer and Lehman, 1993, J. Virology 67(2):711-715; Skaliter and Lehman, 1994, Proc. Natl. Acad. Sci. USA 91(22):10665-10669); single-stranded DNA binding proteins (S S B; Bigler and Romano, 1995, J. Biol. Chem. 270:8910-8919); phage T4 gene 32 protein (Villemain and Giedroc, 1996, Biochemistry 35:14395-14404; and calf thymus helicase (Siegel et al., 1992, J. Biol. Chem. 267:13629-13635).

The ability of a polymerase to carry out strand displacement replication can be determined by using the polymerase in a strand displacement replication assay. Such assays should be performed at a temperature suitable for optimal activity for the enzyme being used, for example, 32° C. for Phi29 DNA polymerase, from 46° C. to 64° C. for exo(−) Bst DNA polymerase, or from about 60° C. to 70° C. for an enzyme from a hyperthermophylic organism. For assays from 60° C. to 70° C., primer length may be increased to 20 bases for random primers, or to 22 bases for specific primers. Another useful assay for selecting a polymerase is the primer-block assay described in Kong et al., 1993, J. Biol. Chem. 268:1965-1975. The assay consists of a primer extension assay using an M13 ssDNA template in the presence or absence of an oligonucleotide that is hybridized upstream of the extending primer to block its progress. Enzymes able to displace the blocking primer in this assay are useful for the disclosed method.

The disclosed method is based on strand displacement replication of the nucleic acid sequences by multiple primers. The method can be used to amplify one or more specific sequences (strand displacement amplification) or other cDNA of high complexity, or circularized cDNA. The method generally involves hybridization of primers to a target nucleic acid sequence and replication of the target sequence primed by the hybridized primers such that replication of the target sequence results in replicated strands complementary to the target sequence. During replication, the growing replicated strands displace other replicated strands from the target sequence (or from another replicated strand) via strand displacement replication. Examples of such displacement of replicated strands are illustrated in U.S. Pat. No. 6,323,009. As used herein, a replicated strand is a nucleic acid strand resulting from elongation of a primer hybridized to a target sequence or to another replicated strand. Strand displacement replication refers to DNA replication where a growing end of a replicated strand encounters and displaces another strand from the template strand (or from another replicated strand). Displacement of replicated strands by other replicated strands is a hallmark of the disclosed method which allows multiple copies of a target sequence to be made in a single, isothermal reaction.

Following amplification, the amplified sequences can be used for any purpose, such as uses known and established for PCR amplified sequences. For example, amplified sequences can be detected using any of the conventional detection systems for nucleic acids such as detection of fluorescent labels, enzyme-linked detection systems, antibody-mediated label detection, and detection of radioactive labels. A key feature of the disclosed method is that amplification takes place not in cycles, but in a continuous, isothermal replication. This makes amplification less complicated and much more consistent in output. Strand displacement allows rapid generation of multiple copies of a nucleic acid sequence or sample in a single, continuous, isothermal reaction. Additionally, amplified sequences can be used to make materials suitable for gene expression analysis. This includes microarray analysis and other quantitative methods used to perform gene expression analysis. Amplification products can also be used to examine alternatively spliced transcripts, edited RNA sequence and for gene cloning experiments.

It is preferred that the set of primers used for SDA have a sequence composition and be used at concentrations that allow the primers to hybridize at desired intervals within the nucleic acid sample. For example, by using a set of primers at a concentration that allows them to hybridize, on average, every 4000 to 8000 bases, DNA replication initiated at these sites will extend to and displace strands being replicated from adjacent sites. It should be noted that the primers are not expected to hybridize to the target sequence at regular intervals. Rather, the average interval will be a general function of primer concentration.

As in strand displacement amplification, displacement of an adjacent strand makes it available for hybridization to another primer and subsequent initiation of another round of replication. The interval at which primers in the set of primers hybridize to the target sequence determines the level of amplification. For example, if the average interval is short, adjacent strands will be displaced quickly and frequently. If the average interval is long, adjacent strands will be displaced only after long runs of replication.

In the disclosed method, the DNA polymerase catalyzes primer extension and strand displacement in a processive strand displacement polymerization reaction that proceeds as long as desired, generating molecules of up to 60,000 nucleotides or larger. Preferred strand displacing DNA polymerases are the DNA polymerase of the bacteriophage Phi29, large fragment Bst DNA polymerase (Exo(−) Bst), exo(−)Bca DNA polymerase and Sequenase. During strand displacement replication one may additionally include radioactive, or modified nucleotides such as bromodeoxyuridine triphosphate, in order to label the DNA generated in the reaction. Alternatively, one may include suitable precursors that provide a binding moiety such as biotinylated nucleotides.

DNA polymerases useful in the rolling circle replication step of rolling circle amplification (RCA) must perform rolling circle replication of primed single-stranded circles (or each strand of a duplex substrate). Such polymerases are referred to herein as rolling circle DNA polymerases. For rolling circle replication, it is preferred that a DNA polymerase be capable of displacing the strand complementary to the template strand, termed strand displacement, and lack a 5′ to 3′ exonuclease activity. Strand displacement is necessary to result in synthesis of multiple tandem copies of the amplification target circle (ATC). Any 5′ to 3′ exonuclease activity, if present, might result in the destruction of the synthesized strand. It is also preferred that DNA polymerases for use in the disclosed method are highly processive. The suitability of a DNA polymerase for use in the disclosed method can be readily determined by assessing its ability to carry out rolling circle replication. Preferred rolling circle DNA polymerases, all of which have 3′,5′-exonuclease activity, are bacteriophage Phi29 DNA polymerase (U.S. Pat. Nos. 5,198,543 and 5,001,050), phage M2 DNA polymerase (Matsumoto et al., 1989, Gene 84:247), phage PRD1 DNA polymerase (Jung et al., 1987, Proc. Natl. Aced. Sci. USA 84:8287), and Zhu and Ito, 1994, Biochim. Biophys. Acta. 1219:267-276), VENT™ DNA polymerase (Kong et al., 1993, J. Biol. Chem. 268:1965-1975), Klenow fragment of DNA polymerase I (Jacobsen et al., 1974, Eur. J. Biochem. 45:623-627), T5 DNA polymerase (Chatterjee et al., 1991, Gene 97:13-19), and T4 DNA polymerase holoenzyme (Kaboord and Benkovic, 1995, Curr. Biol, 5:149-157) φ29 DNA polymerase is most preferred. Equally preferred polymerases include T7 native polymerase, Bacillus stearothermophilus (Bst) DNA polymerase, Thermoanaerobacter thermohydrosulfuricus (Tts) DNA polymerase (U.S. Pat. No. 5,744,312), and the DNA polymerases of Thermus aquaticus, Thermus flavus or Thermus thermophilus. Equally preferred are the Phi29-type DNA polymerases, which are chosen from the DNA polymerases of phages: Phi29, Cp-1, PRD1, Phi15, Phi21, PZE, PZA, Nf, M2Y, B103, SF5, GA-1, Cp-5, Cp-7, PR4, PR5, PR722, and L17. In a specific embodiment, the DNA polymerase is bacteriophage Phi299 DNA polymerase wherein the multiple primers are resistant to exonuclease activity and the target DNA is linear DNA, especially high molecular weight and/or complex linear cDNA.

Strand displacement during RCA can be facilitated through the use of a strand displacement factor, such as a helicase. In general, any DNA polymerase that can perform rolling circle replication in the presence of a strand displacement factor is suitable for use in the processes of the present invention, even if the DNA polymerase does not perform rolling circle replication in the absence of such a factor. Strand displacement factors useful in RCA include BMRF1 polymerase accessory subunit (Tsurumi et al., 1993, J. Virology 67(12):7648-7653), adenovirus DNA-binding protein (Zijderveld and van der Vliet, 1994, J. Virology 68(2):1158-1164), herpes simplex viral protein ICP8 (Boehmer and Lehman, 1993, J. Virology 67(2):711-715); Skaliter and Lehman, 1994, Proc. Natl. Acad. Sci. USA 91(22):10665-10669), single-stranded DNA binding proteins (S S B; Rigler and Romano, 1995, J. Biol. Chem. 270:8910-8919 ( )), and calf thymus helicase (Siegel et al., 1992, J. Biol. Chem. 267:13629-13635).

The ability of a polymerase to carry out rolling circle replication can be determined by testing the polymerase in a rolling circle replication assay such as those described in Fire and Xu, 1995, Proc. Natl. Acad. Sci. USA 92:4641-4645 and in Lizardi (U.S. Pat. No. 5,854,033, e.g., Example 1 therein).

### 2. Detection of Amplification Products

Amplification products can be detected directly by, for example, primary labeling or secondary labeling, as described below.

a. Primary Labeling

Primary labeling consists of incorporating labeled moieties, such as fluorescent nucleotides, biotinylated nucleotides, digoxygenin-containing nucleotides, or bromodeoxyuridine, during strand displacement replication. For example, one may incorporate cyanine dye UTP analogs (Yu et al., 1994, Nucleic Acids Res. 22:3226-3232) at a frequency of 4 analogs for every 100 nucleotides. A preferred method for detecting nucleic acid amplified in situ is to label the DNA during amplification with BrdUrd, followed by binding of the incorporated BUDR with a biotinylated anti-BUDR antibody (Zymed Labs, San Francisco, Calif.), followed by binding of the biotin moieties with Streptavidin-Peroxidase (Life Sciences, Inc.), and finally development of fluorescence with Fluorescein-tyramide (DuPont de Nemours & Co., Medical Products Dept.).

b. Secondary Labeling with Detection Probes

Secondary labeling consists of using suitable molecular probes, referred to as detection probes, to detect the amplified DNA or RNA. For example, a primer may be designed to contain, in its non-complementary portion, a known arbitrary sequence, referred to as a detection tag. A secondary hybridization step can be used to bind detection probes to these detection tags. The detection probes may be labeled as described above with, for example, an enzyme, fluorescent moieties, or radioactive isotopes. By using three detection tags per primer, and four fluorescent moieties per each detection probe, one may obtain a total of twelve fluorescent signals for every replicated strand.

c. Enzyme-Linked Detection

Amplified nucleic acid labeled by incorporation of labeled nucleotides can be detected with established enzyme-linked detection systems. For example, amplified nucleic acid labeled by incorporation of biotin-16-UTP (Boehringher Mannheim) can be detected as follows. The nucleic acid is immobilized on a solid glass surface by hybridization with a complementary DNA oligonucleotide (address probe) complementary to the target sequence (or its complement) present in the amplified nucleic acid. After hybridization, the glass slide is washed and contacted with alkaline phosphatase-streptavidin conjugate (Tropix, Inc., Bedford, Mass.). This enzyme-streptavidin conjugate binds to the biotin moieties on the amplified nucleic acid. The slide is again washed to remove excess enzyme conjugate and the chemiluminescent substrate CSPD (Tropix, Inc.) is added and covered with a glass cover slip. The slide can then be imaged in a Biorad Fluorimager.

### E. Linear Strand Displacement Amplification

A modified form of strand displacement amplification can be performed which results in linear amplification of a target sequence. This modified method is referred to as linear strand displacement amplification (LSDA) and is accomplished by using a set of primers where all of the primers are complementary to the same strand of the target sequence. In LSDA, as in MSDA, the set of primers hybridize to the target sequence and strand displacement amplification takes place. However, only one of the strands of the target sequence is replicated. LSDA requires thermal cycling between each round of replication to allow a new set of primers to hybridize to the target sequence. Such thermal cycling is similar to that used in PCR. Unlike linear, or single primer, PCR, however, each round of replication in LSDA results in multiple copies of the target sequence. One copy is made for each primer used. Thus, if 20 primers are used in LSDA, 20 copies of the target sequence will be made in each cycle of replication.

DNA amplified using MSDA can be further amplified by transcription. For this purpose, promoter sequences can be included in the non-complementary portion of primers used for strand displacement amplification.

### F, PCR

The components of a typical PCR reaction include but are not limited to a template DNA, primers, a reaction buffer (dependent on choice of polymerase), dNTPs (dATP, dTTP, dGTP, and dCTP) and a DNA polymerase. Suitable PCR primers can be designed and prepared as discussed above (see “Primer Design” section above). Briefly, the reaction is heated to 95° C. for 2 minutes. to separate the strands of the template DNA, the reaction is cooled to an appropriate temperature (determined by calculating the annealing temperature of designed primers) to allow primers to anneal to the template DNA, and heated to 72° C. for two minutes to allow extension.

In one embodiment, the annealing temperature (TM) is increased in each of the first three cycles of amplification to reduce non-specific amplification. The TM of the first cycle of PCR (TM1) is about the melting temperature of the 3′region of the second primer that anneals to the template DNA. The annealing temperature can be raised in cycles 2-10, preferably in cycle 2, to TM2, which is about the melting temperature of the 3′ region, which anneals to the template DNA, of the first primer. If the annealing temperature is raised in cycle 2, the annealing temperature remains about the same until the next increase in annealing temperature. Finally, in any cycle subsequent to the cycle in which the annealing temperature was increased to TM2, preferably cycle 3, the annealing temperature is raised to TM3, which is about the melting temperature of the entire second primer. After the third cycle, the annealing temperature for the remaining cycles can be at about TM3 or can be further increased. In this example, the annealing temperature is increased in cycles 2 and 3. However, the annealing temperature can be increased from a low annealing temperature in cycle I to a high annealing temperature in cycle 2 without any further increases in temperature or the annealing temperature can progressively change from a low annealing temperature to a high annealing temperature in any number of incremental steps. For example, the annealing temperature can be changed in cycles 2, 3, 4, 5, 6, etc.

After annealing, the temperature in each cycle is increased to an “extension” temperature to allow the primers to “extend” and then following extension the temperature in each cycle is increased to the denaturization temperature. For PCR products less than 500 base pairs in size, one can eliminate the extension step in each cycle and just have denaturization and annealing steps. A typical PCR reaction consists of 25-45 cycles of denaturation, annealing and extension as described above. However, as previously noted, one cycle of amplification (one copy) can be sufficient for practicing the invention.

In another embodiment, multiple sets of nested primers wherein a primer set comprises a forward primer and a reverser primer, can be used to amplify the loci of interest on template DNA for 1-5, 5-10, 10-15, 15-20 or more than 20 cycles. In one embodiment, the amplified product may be further amplified in a reaction with a single primer set or a subset of the multiple primer sets. In a preferred embodiment, a low concentration of each primer set is used to minimize primer-dimer formation. A low concentration of starting DNA can be amplified using multiple primer sets.

The multiple primer sets will amplify the loci of interest, such that a minimal amount of template DNA is not limiting for the number of loci that can be detected. For example, if template DNA is isolated from a single cell or the template DNA is obtained from a pregnant female, which comprises both maternal template DNA and fetal template DNA, low concentrations of each primer set can be used in a first amplification reaction to amplify the loci of interest. The low concentration of primers reduces the formation of primer-dimer and increases the probability that the primers will anneal to the template DNA and allow the polymerase to extend. The optimal number of cycles performed with the multiple primer sets is determined by the concentration of the primers. Following the first amplification reaction, additional primers can be added to further amplify the loci of interest. Additional amounts of each primer set can be added and further amplified in a single reaction. Alternatively, the amplified product can be further amplified using a single primer set in each reaction or a subset of the multiple primers sets. For example, if 150 primer sets were used in the first amplification reaction, subsets of 10 primer sets can be used to further amplify the product from the first reaction.

Any DNA polymerase that catalyzes primer extension can be used including but not limited to E. coli DNA polymerase, Klenow fragment of E. coli DNA polymerase 1, T7 DNA polymerase, T4 DNA polymerase, Taq polymerase, Pfu DNA polymerase, Vent DNA polymerase, bacteriophage 29, REDTaq™ Genomic DNA polymerase, or sequenase. Preferably, a thermostable DNA polymerase is used. A “hot start” PCR can also be performed wherein the reaction is heated to 95° C. for two minutes prior to addition of the polymerase or the polymerase can be kept inactive until the first heating step in cycle 1. “Hot start” PCR can be used to minimize nonspecific amplification. Any number of PCR cycles can be used to amplify the DNA, including but not limited to 2, 5, 10, 15, 20, 25, 30, 35, 40, or 45 cycles. In a most preferred embodiment, the number of PCR cycles performed is such that equimolar amounts of each loci of interest are produced.

### III. Kits

The invention encompasses various kits relating to screening, identifying and/or diagnosing a subject for a triplet repeat genetic disorder, including fragile X syndrome. The present invention further comprises a method of screening for and diagnosing a human subject at-risk of having progeny with a triplet repeat genetic disorder.

The kits of the present invention can be used to perform population screening or individual screening of a newborn, a fetus, or a child. The kit of the present invention can comprise primers that specifically bind to regions of the FMR1 gene disclosed elsewhere herein for diagnosis of fragile X syndrome in various clinical labs. The present invention further comprises kits for the collection of a biological sample. A patient or practitioner can collect a biological sample and send the sample to a clinical lab where the present screen for fragile X syndrome is performed.

The present invention further comprises DNA collection kits for detecting a triplet repeat genetic disorder including fragile X syndrome. The kits of the present invention can comprise reagents and materials to expedite the collection of samples for DNA extraction and analysis. These kits can comprise an intake form with a unique identifier, such as a bar-code, a sterile biological collection vessel, such as a Catch-All™ swab (Epicentre® Madison, Wis.) for collecting loose epithelial cells from inside the cheek; and an instruction material that depicts how to properly apply the swab, dry it, repack it and return to a clinical lab. The kit can further comprise a return postage-paid envelope addressed to the clinical lab to facilitate the transport of biological samples.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

At present, it is estimated that more than 100,000 individuals are tested for FRAX per year in the United States. As practitioners become more aware of FRAX, and FRAX testing is expanded to children with autism (1 in 150 children; 300,000 children born per year with autism spectrum), it is anticipated that at least 500,000 individuals would be tested for FRAX if a low cost test is made available.

Presently, Southern Blot analysis is used to determine the size of the repeat segment and methylation status of the FRAX gene. This test only detects the gross size of CGG repeats and is labor intensive and expensive. PCR and gel electrophoresis is typically used to determine the size of the CGG expansion. This approach is limited, as PCR reactions typically fail to amplify long stretches of CGG expansions (>25 repeats) and molecular weight determination by electrophoresis via capillary or slab gels is labor intensive. In addition, FRAX testing is expensive. As the result, many individuals who should be screened for FRAX are not tested for this condition.

It is currently recommended that genetic testing for FRAX should be considered in, but not limited to, the following indications: 1) Children (males and females) with developmental, speech, language or motor delay; children with a diagnosis of learning disabilities of unknown etiology, autism, autistic spectrum disorder, pervasive developmental disorder (PDD) or mental retardation; adults with mental retardation or autism of unknown cause. 2) Individuals with a family history of FRAX to determine if they may be carriers and at risk of transmitting it to future generations. 3) Individuals with a family history of mental retardation or autism of unknown cause. 4) Women who are known carriers and pregnant can meet with a genetic counselor to discuss prenatal testing to determine if the fetus carries the gene mutation, 5) Individuals over 50 years old with tremors, balance disorders, or Parkinsonian-like findings without a diagnosis can be tested for a condition called FXTAS (fragile X-associated tremor ataxia syndrome). FXTAS is caused by changes in the same gene that causes fragile X syndrome and has been primarily described in individuals over 50 with a premutation in the fragile X gene.

### Example 1

### Site Specific Multiple Displacement Amplification (SSMDA) to Amplify and Enrich the 5′ Untranslated Region of FRAX Associated FMR1 Gene

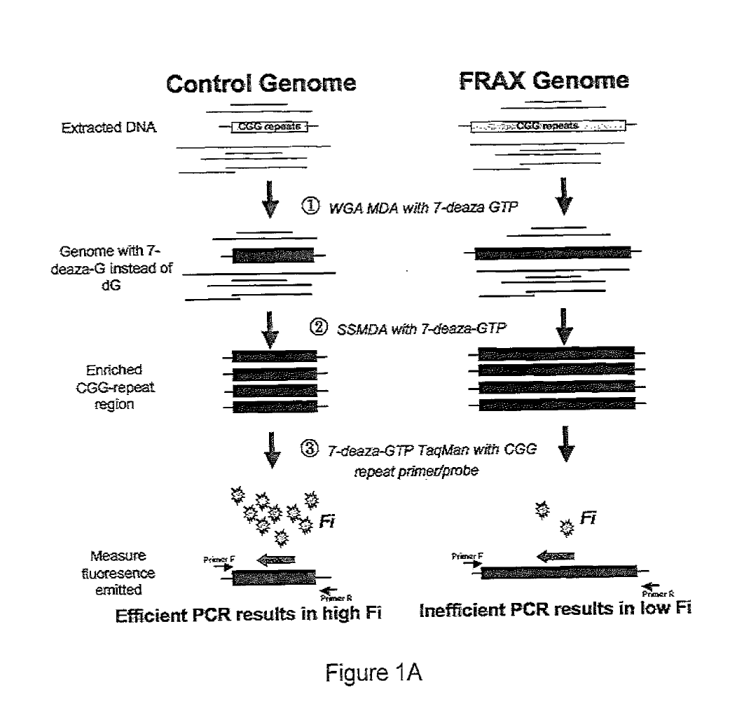

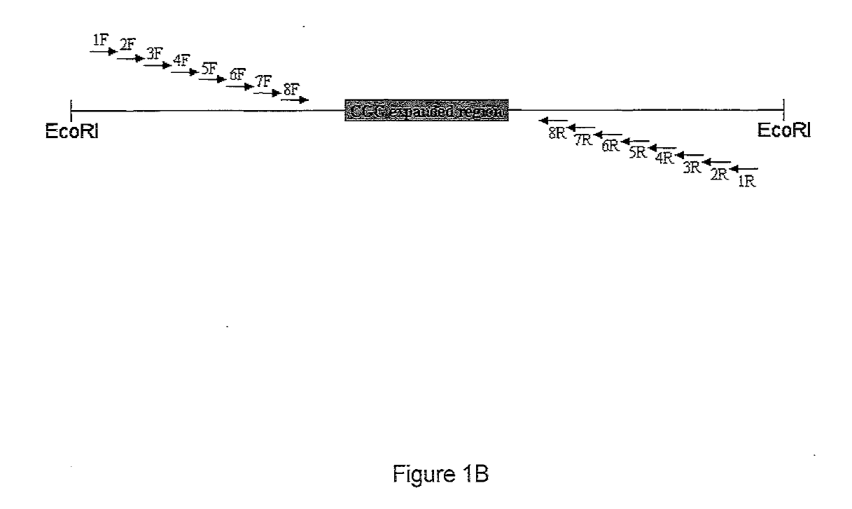

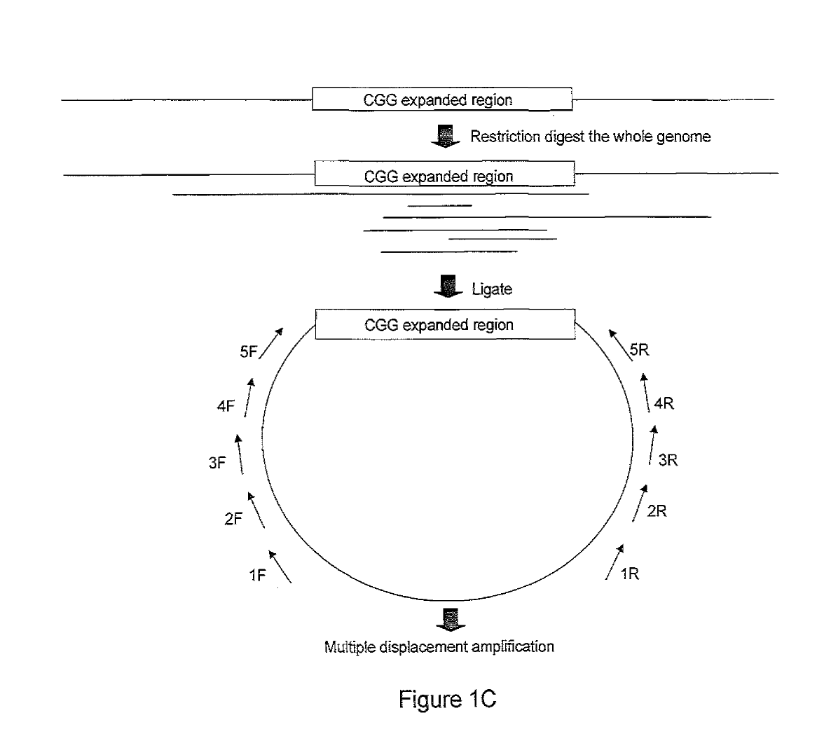

Considering the limitation of prior art methods to assay for FRAX, there is a need to develop a highly accurate, inexpensive, automated, and high-throughput test for diagnosing FRAX. The results presented herein demonstrate the development of a novel approach to screen for FMR1:CGG triplet repeat expansion by way of amplification using Whole Genome Amplification (MDA) and Sequence Specific Amplification with Multiple Displacement Amplification (SSMDA), followed by quantitative assessment of the number of CGG repeats using TaqMan real-time Polymerase Chain Reaction of the SSMDA-enriched 5′ untranslated region of the FMR1 gene. This novel method utilizes, for example, a dGTP nucleotide analog 7-deaza-GTP, which helps alleviate the strong hydrogen bonding of the GC rich region (FIG. 1A). The resulting fluorescence endpoint signal can be measured in a highly quantitative manner using a CGG-repeat specific TaqMan probe.

The materials and methods employed in the experiments disclosed herein are now described.

### Isothermal Multiple Displacement Amplification (MDA)

PCR is an efficient method for DNA amplification in DNA diagnostics. Due to the formation of secondary hairpin structures and the requirement for higher melting temperatures (Tₘ), PCR amplification is poor when the target sequence is highly GC-rich. This problem of amplification is compounded when the PCR target sequence becomes longer, as the number of GC-repeats increase.

An alternative method of whole genome amplification technology applicable to the present invention is called “Multiple Displacement Amplification” (MDA). MDA efficiently amplifies the entire human genome uniformly, even through highly GC-rich, regardless of the target length. Whole genome amplification (WGA) by MDA utilizes the Phi29 DNA polymerase and random primers to amplify the entire genome to up to 10⁶-fold at 30° C., starting with nanogram amounts of genomic DNA. MDA has been applied to small amounts of genomic DNA samples, leading to whole genome amplification of high molecular weight DNA with minimal sequence representation bias.

Amplification is made possible by the strand displacing activity and the high enzyme processivity of Phi29 DNA polymerase, which synthesizes DNA strands up to 70 kb in length. Importantly, proofreading activity of the Phi29 polymerase ensures high-fidelity amplification with an error rate of 3×10⁻⁶ mutations per nucleotide in the amplified DNA. This error rate is markedly better than the rate of 1×10⁻³ mutations per nucleotide generated by Taq DNA polymerase in PCR reactions. MDA-based whole genome amplification creates minimal amplification bias along the entire genome, as compared with PCR-based methods.

### Quantitative Real-Time Polymerase Chain Reaction (TaqMan)

One of the most widely used PCR-based diagnostic technologies is “TaqMan”. TaqMan is a fluorophore-based Real-Time PCR (RT-PCR), which is highly quantitative and suitable for high-throughput screening. TaqMan Real-time PCR can measure the accumulation of a product via fluorescent reporter probes that anneal to a region between the two PCR primers during the exponential stages of the PCR reaction. The exponential increase of the reaction product is used to determine the threshold cycle (Cₜ value), which is the number of PCR cycles at which a significant exponential increase in fluorescence is detected. This value correlates directly with the number of copies of DNA template present in the reaction.

The TaqMan fluorescent reporter probes consist of a fluorophore, such as 6-carboxyfluorescein (FAM), and a quencher, such as dihydrocyclopyrroloindole tripeptide (MGB). The probe and quencher are covalently attached to the 5′ and 3′ ends of the probe, respectively. The close proximity between the fluorophore and the quencher inhibits fluorescence from the fluorophore. As DNA synthesis occurs during PCR, the 5′ to 3′ exonuclease activity of the Taq DNA polymerase degrades that portion of the probe that has annealed to the template. Degradation of the probe releases the fluorophore and breaks the close proximity to the quencher. This phenomenon releases the quenching effect allowing fluorescence of the fluorophore. The fluorescence detected by real-time PCR thermal cyclers is directly proportional to the amount of fluorophore released and the amount of DNA template present.

TaqMan is highly automatable, since it is carried out in a real-time thermal cycler with built-in fiber optic cables that measure the fluorescence in the reaction tubes using laser beams for excitation and detection of the fluorescent emission from the fluorophore. This process allows direct measurement of fluorescent molecules in PCR tubes. Fluorescence intensities are recorded. The data stored after each PCR cycle are used to create amplification plots of ΔRn (fluorescent signal detected—background) vs. cycle number. These data identify the threshold cycle, Ct, which is used to quantitatively determine the amount of DNA template present in the PCR reaction.

The results of these experiments are now described.

### Develop Specific Multiple Displacement Amplification (SSMDA) Reaction Conditions

The commonly used polymerase chain reaction (PCR) cannot amplify a GC-rich region in DNA with consistency. To overcome this, site specific multiple displacement amplification (SSMDA) was used. SSMDA is a modification of multiple displacement amplification (MDA) which is a non-PCR isothermal method based on the annealing of random hexamers to denatured DNA, followed by strand-displacement synthesis at constant temperature. SSMDA has been applied to small amounts of genomic DNA samples, leading to whole genome amplification of high molecular weight DNA with minimal sequence representation bias. As DNA is synthesized by strand displacement, a gradually increasing number of priming events occur, forming a network of hyper-branched DNA structures. The reaction is performed by the phi29 DNA polymerase, which possesses a proofreading activity resulting in error rates 100 times lower than the Taq polymerase and a high enzyme processivity, which allows the amplification of a DNA region previously not possible with Taq DNA Polymerase (PCR) like the GC-rich region of the triplet repeat region of fragile X syndrome associated FMR1 gene.

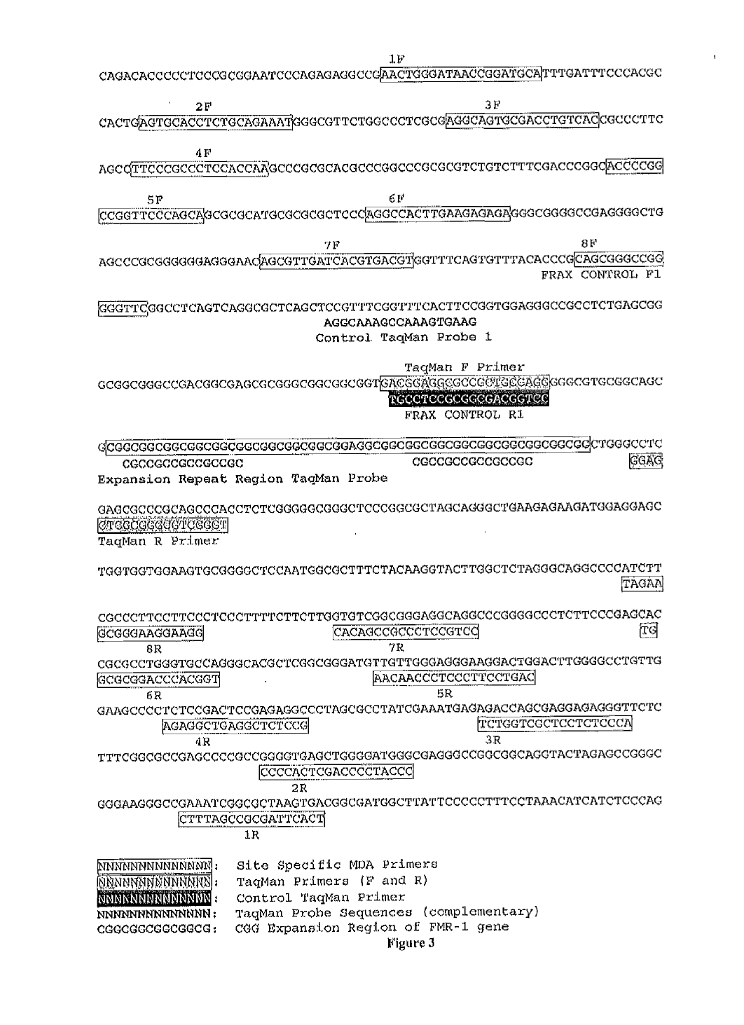

Two methods are encompassed in the present invention. FIG. 1B illustrates the first mechanism of SSMDA reaction to amplify and enrich the 5′ untranslated region of the FMR1 gene for subsequent analysis (FIG. 3). SSMDA is performed using 5-10 pairs of nested primers (e.g. Table 1) flanking the region of interest. Each primer is 15-20 nucleotides long. Primer concentration during the SSMDA reaction is 200 nM each. Reaction conditions using Phi29 is as follows (Dean et al., 2002, Proc. Natl. Acad. Sci. 99:5261-6). Primers are thiol-protected at the last two nucleotides of the 3′ end to protect from endonuclease activity of Phi29 DNA polymerase. Distance between the primers is about 20 base pairs, DNA (300 ng to 0.03 ng) is placed into 0.2 ml tubes in a total volume of 100 μl containing 37.5 mM Tris-HCl (pH 7.5), 50 mM KCl, 10 mM MgCl₂, 20 mM (NH₄)₂SO₄, 1 mM dATP, dCTP, dTTP and dGTP (dGTP can be replaced by either dITP or deaza-GTP), 200 nM each of exonuclease-resistant primer sets as depicted in Table 1 and 800 units/ml Phi29 DNA polymerase. Reactions were incubated for 18 hours at 30° C. and terminated by heating to 65° C. for 3 minutes.

FIG. 1C illustrates the second mechanism of SSMDA reaction to amplify and enrich the 5′ untranslated region of the FMR1 gene for subsequent analysis. Genomic DNA is digested with a 6-cutter restriction enzyme(s), which digests flanking the region with 5-10 sets of primers (Table 1). Both Eco RI and Xho I are ideal restriction enzyme which cuts just outside of the primer annealing region. The digested DNA is diluted and ligated using DNA ligase in order to facilitate self ligation to occur (monomolecular ligation event). The resulting circular DNA with the target sequence of FMR1 gene is amplified by SSMDA performed using 5-10 pairs of nested primers (Table 1)). The reaction conditions are exactly same as those described FIG. 1B.

Thirty one (31) commercially available FRAX and non-FRAX genomic DNA samples with known number of CGG repeats (Table 2) were examined. The results indicate that ALL male samples with premutation or full mutation of the CGG expanded region of the FMR-1 gene were definitively identify.

TABLE 2
FRAX and non-FRAX samples tested.

| # | Reposit# | Repeat# | SexGender | Phenotype | FRAX Class |
|---|---|---|---|---|---|
| 1 | NA04025 | 645 | Male | FRAX | Full mutation |
| 2 | NA06852 | >200 | Male | FRAX | Full mutation |
| 3 | NA06897 | 477 | Male | FRAX | Full mutation |
| 4 | NA07365 | >200 | Male | FRAX | Full mutation |
| 5 | NA09145 | >200 | Male | FRAX | Full mutation |
| 6 | NA06911 | 30, >200 | Female | normal | Carrier Full |
| | | | | | mutation |
| 7 | NA07537 | 28, >200 | Female | normal | Carrier Full |
| | | | | | mutation |
| 8 | NA06891 | 118 | Male | normal | Premutation |
| 9 | NA06892 | 93 | Male | normal | Premutation |
| 10 | NA06906 | 96 | Male | normal | Premutation |
| 11 | NA06893 | 23, 95 | Female | normal | Carrier |
| | | | | | Premutation |
| 12 | NA06894 | 30, 78 | Female | normal | Carrier |
| | | | | | Premutation |
| 13 | NA06896 | 23, 120 | Female | normal | Carrier |
| | | | | | Premutation |
| 14 | NA06903 | 23, 95 | Female | normal | Carrier |
| | | | | | Premutation |
| 15 | NA06905 | 23, 70 | Female | normal | Carrier |
| | | | | | Premutation |
| 16 | NA06907 | 29, 85 | Female | normal | Carrier |
| | | | | | Premutation |
| 17 | NA06968 | 23, 107 | Female | normal | Carrier |
| | | | | | Premutation |
| 18 | NA13664 | 28, 49 | Female | normal | Carrier |
| | | | | | Intermediate |
| 19 | NA06890 | 30 | Male | normal | Normal |
| 20 | NA06895 | 23 | Male | normal | Normal |
| 21 | NA07174 | 30 | Male | normal | Normal |
| 22 | NA07536 | 23 | Male | normal | Normal |
| 23 | NA07539 | 23 | Male | normal | Normal |
| 24 | NA07542 | 23 | Male | normal | Normal |
| 25 | NA06889 | 23, 30 | Female | normal | Normal |
| 26 | NA06904 | 23, 29 | Female | normal | Normal |
| 27 | NA07175 | 23, 30 | Female | normal | Normal |
| 28 | NA07538 | 29, 29 | Female | normal | Normal |
| 29 | NA07540 | 23, 29 | Female | normal | Normal |
| 30 | NA07541 | 29, 31 | Female | normal | Normal |
| 31 | NA07543 | 20, 29 | Female | normal | Normal |

Step 1: Conversion of Genomic DNA into a 7-deaza-Guanine Genome by Whole Genome Amplification (WGA)

The most challenging obstacle in the development of a highly accurate and high-throughput DNA-based molecular diagnostic procedure suitable for FRAX is difficulty in reliably detecting CGG expanded repeats in the 5′UTR of FMR1. This problem is due to the high GC-content of the expanded region that impedes efficient and reliable detection by PCR amplification. To overcome this problem, experiments were performed using an MDA-based WGA technology, substituting dGTP with a nucleotide analog 7-deaza-2-deoxyguanosine (7-deaza-GTP), which converts the genome into one which has a weaker hydrogen bonding with cytosine. 7-deaza-G to C pairing has only two hydrogen bonds compared to three hydrogen bonds in a normal G-C pairing. The reaction is performed in a single tube (FIG. 1A).

Briefly, genomic DNA is amplified by MDA. 7-deaza-GTP is used instead of dGTP using a random hexamer with thiophosphate group conjugated to the final two nucleotides of the 3′ terminus to protect them from Phi29 DNA polymerase 3′ exonuclease activity. Phi29 DNA Polymerase (New England Biolabs), and genomic DNA are then incubated at 30° C. for 16 hrs. The reaction time is the duration it takes to achieve a saturation concentration of 1 μg/μl, for the amplified product, which was confirmed by Picogreen fluorescence assay (Molecular Probes). Completion of this step results in genomic DNA that is suitable for amplification.

### Step 2: Optimization of Site Specific Multiple Displacement Amplification (SSMDA) of FMR1

The next set of experiments were designed to optimize SSMDA methods by modifying MDA technology for FRAX testing. Genomic DNA (from Step 1) was amplified by SSMDA using a nucleotide analog 7-deaza-GTP instead of dGTP and 8 pairs of nested oligonucleotide primers (1F, 2F, 3F, 4F, 5F, 6F, 7F, 8F and 1R, 2R, 3R, 4R, 5R, 6R, 7R, 8R). Nested primer sequences and locations are shown in FIGS. 1 and 3.

In this step, the 7-deaza-Guanine converted genomic DNA (from Step 1) is transferred to a fresh tube for the SSMDA reaction along with MDA buffer. SSMDA is performed using 7-deaza-GTP, 8 pairs of nested primers flanking the 5′UTR of FMR1 (FIGS. 1 and 3), and Phi29 DNA Polymerase (New England Biolabs). The primers were designed by us to be 15-20 nucleotides long, with thiophosphate linked at the two 3′ terminal nucleotides to protect them from Phi29 DNA polymerase's 3′ exonuclease activity. The distance between primers is about 20 base pairs (FIGS. 1 and 3). The reaction time is approximately the time it takes to achieve a saturation concentration of 1 μg/μL (±5%) for the amplified product, as confirmed by Picogreen fluorescence assay (Molecular Probes), making SSMDA suitable for high-throughput automation.

### Step 3: Analysis of SSMDA Amplified DNA Products by TaqMan Quantitative PCR Assay

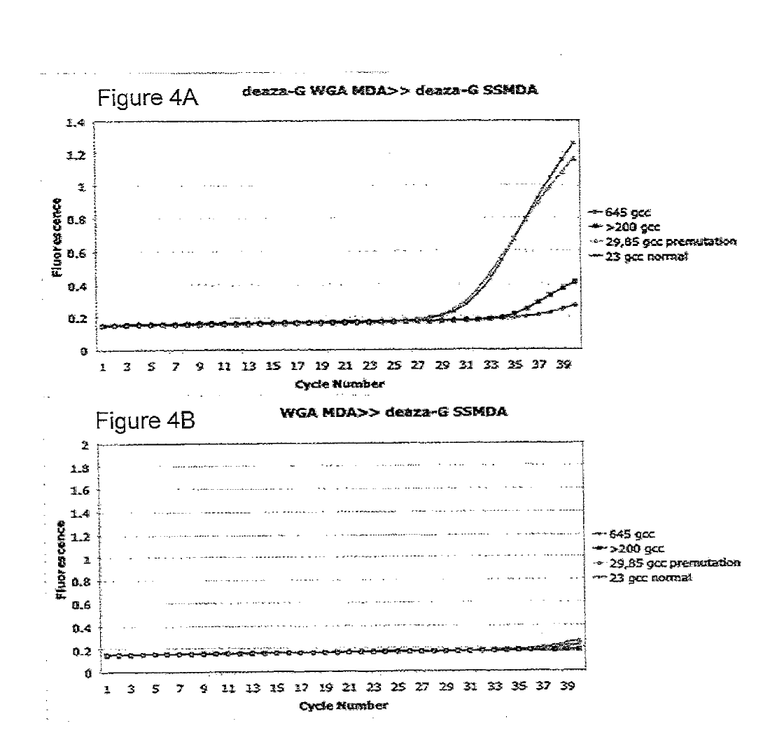

The next experiments were designed to utilize the TaqMan assay for confirming the presence or absence of expanded CGG repeat sequences (FIGS. 1 and 4). To test our approach, the amplification efficiency between FRAX and non-FRAX DNA samples was compared by a real-time quantitative TaqMan PCR method using a TaqMan probe 5′FAM-(CGCCGCCGCCGCCGC)-MGB′3 probe (SEQ ID NO: 21).

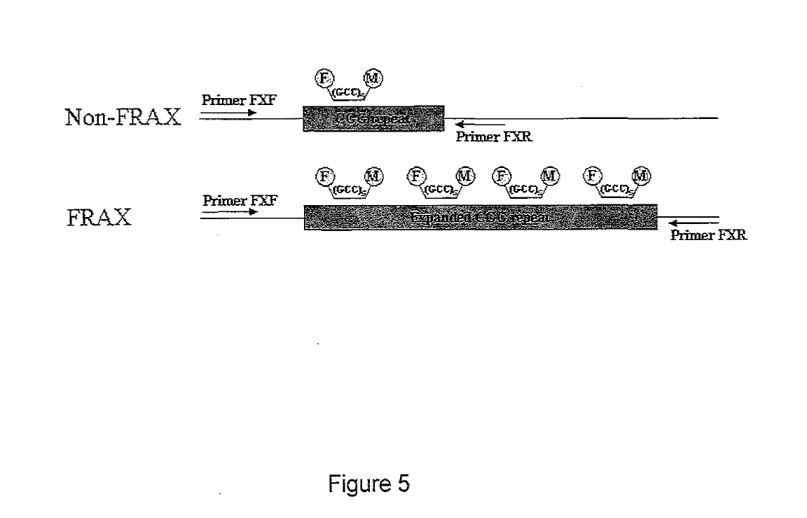

After extensive modification and optimization of reaction conditions, an assay in which the expanded CGG repeat number can be accurately and consistently measured in an inversely proportional manner to the amplification endpoint signal intensity was established (FIGS. 4 and 5). For example, a non-FRAX male DNA sample (XY 23 repeats) and heterozygous female sample with one allele with normal copy number (XX 29 and 85 repeats) can be detected in a consistent manner (FIG. 4). This discrimination is due to the presence of the unexpanded 23 repeat allele which allows efficient amplification resulting in a strong endpoint fluorescence intensity of about 1.2 units after 40 cycles. In comparison, FRAX DNA samples with 645 and >200 repeats, respectively showed delayed and inefficient amplification, allowing clear differences between normal and FRAX samples to be detected.

### Experiment 2

### Using Real-Time SSMDA or PCR to Quantify the Number of Triplet Codon Repeats from the Enriched 5′ Untranslated Region of Fragile X Syndrome Associated FMR1 Gene

In the above studies, it was observed that individuals with FRAX can be distinguish from controls using a TaqMan real-time PCR assay using a CGG repeat-specific probe. The next set of experiments were designed to determine the optimal conditions for the TaqMan PCR reaction to assess CGG repeat numbers from the SSMDA reaction products.

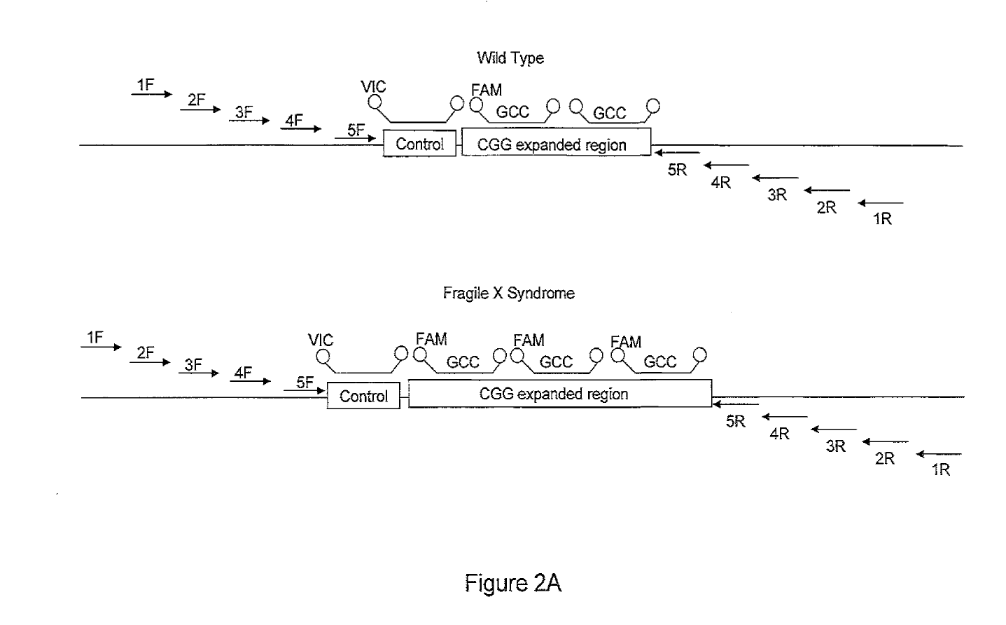

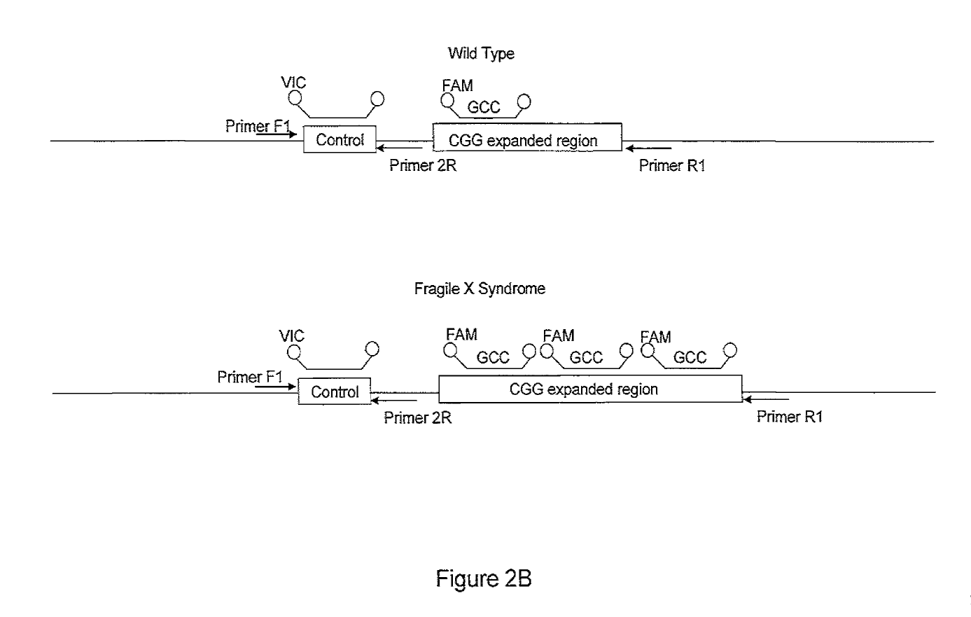

After the enrichment of the 5′ untranslated region of FMR1 gene is accomplished by SSMDA, the next step is to quantify the number of CGG repeat found in the amplified region. Two possible protocols may be used. FIG. 2A illustrates the first methods to quantify the number of triplet codon repeats from the 5′ untranslated region of fragile X syndrome associated FMR1 gene. 5′FAM-(CGC)₅-TAMRA′3 (SEQ ID NO, 20) or 5′FAM-(CGC)₅-MGB′3 (SEQ ID NO, 21) is used as a fluorescent probe to detect the CGG expansion. 5′VIC-(GAAGTGAAACCGAAACGGA)-TAMRA′3 (SEQ ID NO. 24) or 5′VIC-(GAAGTGAAACCGAAACGGA)-MGB′3 (SEQ ID NO. 25) which is a sequence upstream of the triplet repeat sequence will be used as a fluorescent probe to detect internal control sequence, which serves as a internal reference. The probes should emit fluorescence relative to the number of copies of the CGG repeats and the internal control. By including the two probes in the SSMDA reaction, one can quantify the amount of fluorescence emitted using a real-time fluorescence reader directly as the triplet repeat region is being amplified by SSMDA using 5-10 pairs of nested primers (1F, 2F, 3F, 4F, 5F and 1R, 2R, 3R, 4R, 5R) flanking the region of interest. Fluorescence signal from a DNA sample containing an expansion of the CGG triplet repeat region of the FMR1 gene should be higher than that from a normal (WT) control DNA.

FIG. 2B illustrates the second mechanism to quantify the number of triplet codon repeats from the “amplified and enriched” 5′ untranslated region of fragile X syndrome associated FMR1 gene. TaqMan real-time PCR reaction is performed using primers depicted in Table 1 as the amplification primers. 5′FAM-(CGC)₅-TAMRA′3 (SEQ ID NO. 20) or 5′FAM-(CGC)₅-MGB′3 (SEQ ID NO. 21) is used as a fluorescent probe to detect the CGG expansion. 5′VIC-(GAAGTGAAACCGAAACGGA)-TAMRA′3 (SEQ ID NO. 24) or 5′VIC-(GAAGTGAAACCGAAACGGA)-MGB′3 (SEQ ID NO. 25) which is a sequence upstream of the triplet repeat sequence will be used as a fluorescent probe to detect internal control sequence, which serves as a internal reference. The GCC-probe should emit FAM fluorescence signal relative to the number of copies of the CGG repeats. Fluorescence signal from DNA with an expansions in the CGG triplet repeat region of the FMR1 gene should be higher than that from a normal (WT) control DNA.

Use of 7-deaza-GTP Instead of dGTP in a TaqMan Reaction

It was determined that the optimal reaction conditions for when dGTP was replaced with 7-deaza-GTP and with FXF and FXR primers (FIGS. 1 and 5). Default thermal cycling parameters used for TaqMan assay (Applied Biosystems) were used.

To detect CGG repeat expansions, a 5′FAM-CGCCGCCGCCGCCGC-MGB′3 probe (SEQ ID NO: 21) was used as a TaqMan fluorescent probe. Primers FXF and FXR were used as the amplification primers situated inside of the inner most SSMDA primer set 1F and 1R (FIGS. 1 and 5). Quantitative real-time TaqMan PCR analysis was performed using the ABI 7500 real-time thermocycler according to the manufacturer's specifications (Applied Biosystems, Foster City, Calif.).

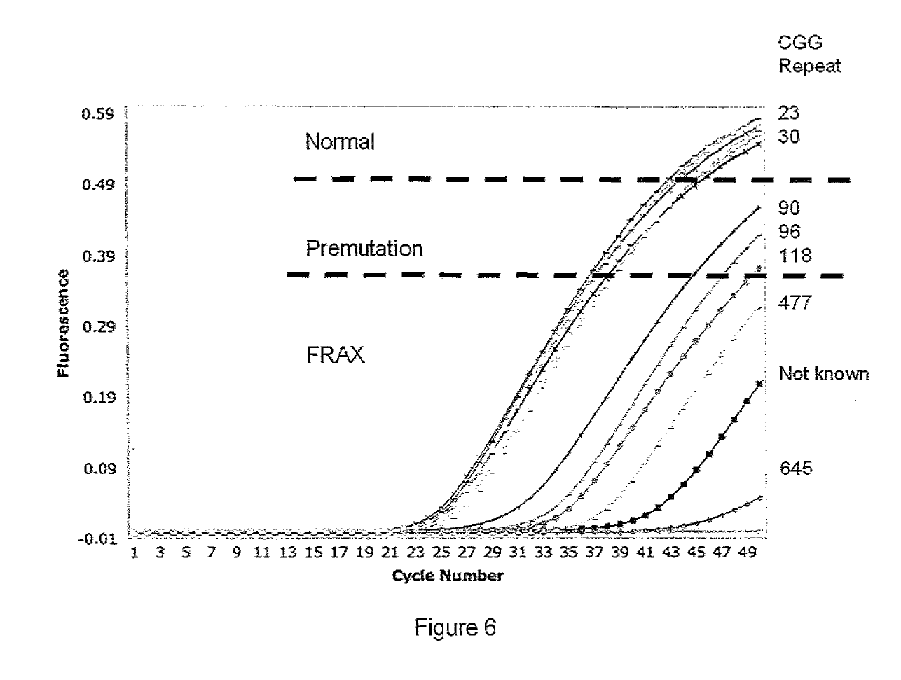

After extensive optimization, it was observed that both the Ct value and the end point fluorescence signal intensity (Fi) after 50 cycles of TaqMan (FIGS. 1 and 6) correlated consistently, in an inversely proportional manner, with the number of CGG repeats. This observation reflects the inefficient PCR of expanded CGG-repeats of FRAX patients. Thus, the endpoint FAM fluorescence signals from non-FRAX unexpanded DNAs are higher than those from FRAX DNA.

The next experiments were designed to establish a quantitative assay system for FRAX with which the extent of COO repeats can be measured using optimized conditions. The 31 FRAX and non-FRAX DNA samples with known length of CGG repeats (Table 2) were studied in triplicate. These data were used to establish algorithms to calculate the CGG repeat lengths.

Five male FRAX genomic DNA samples were studied with “full mutation” (samples 1-5). Three male samples had varying degrees of “premutation” (samples 8-10). Ten female were identified as heterozygous with either premutation or full mutation (sample 6, 7, 11-18). In addition, 13 non-FRAX, normal control (samples 19-31) were identified with a known number of CGG repeats.

By testing different PCR primer set combinations, it was found that primers between Forward I and Reverse 4 showed the cleanest TaqMan/PCR product without any nonspecific product or primer dimers. We also tested different thermocycling conditions. Instead of using the default thermal cycling parameters for conventional TaqMan assays (Applied Biosystems), we found that the by modifying the parameters to 95° C./15 sec, 60° C./1 min and 72° C./1 min, for 50 cycles, we could differentiate between the various length of CGG repeats (FIG. 6; Table 3).

TABLE 3
Ct values (Ct) of various FRAX and non-FRAX samples.

| | Coriell | | | | | Average | Std Dev |
|---|---|---|---|---|---|---|---|
| # | Reposit# | Repeat# | Gender | Phenotype | FRAX Class | Ct Value | Ct Value |
| 1 | NA04025 | 645 | Male | FRAX | Full mutation | 43.859 | 1.317 |
| 2 | NA06852 | Unknown | Male | FRAX | Full mutation | 39.989 | 0.106 |
| 3 | NA06897 | 477 | Male | FRAX | Full mutation | 36.348 | 0.589 |
| 4 | NA06891 | 118 | Male | normal | Premutation | 32.965 | 0.431 |
| 5 | NA06906 | 96 | Male | normal | Premutation | 31.795 | 0.156 |
| 6 | NA06892 | 93 | Male | normal | Premutation | 28.505 | 0.636 |
| 7 | NA07536 | 23 | Male | normal | Normal | 24.964 | 0.030 |
| 8 | NA06889 | 23, 30 | Female | normal | Normal | 24.638 | 0.027 |
| 9 | NA07538 | 29, 29 | Female | normal | Normal | 24.103 | 0.261 |

### Example 3

### Establish Methods to Estimate CGG Copy Number in FRAX

With the background provided by the above studies, the next set of experiments was designed to examine thresholds to distinguish between various size CGG-repeats. Values for average Ct value (Ct) and standard deviation of duplicate experiments are presented in elsewhere herein. All male FRAX DNA with full mutations above 477 had Ct values above 36 cycles and premutations between 93 and 118 CGG repeats had Ct values between 28 and 33. The male non-FRAX DNAs and females with at least one normal 23 or 30 CGG with normal 23 or 30 repeats had Ct values below 25.

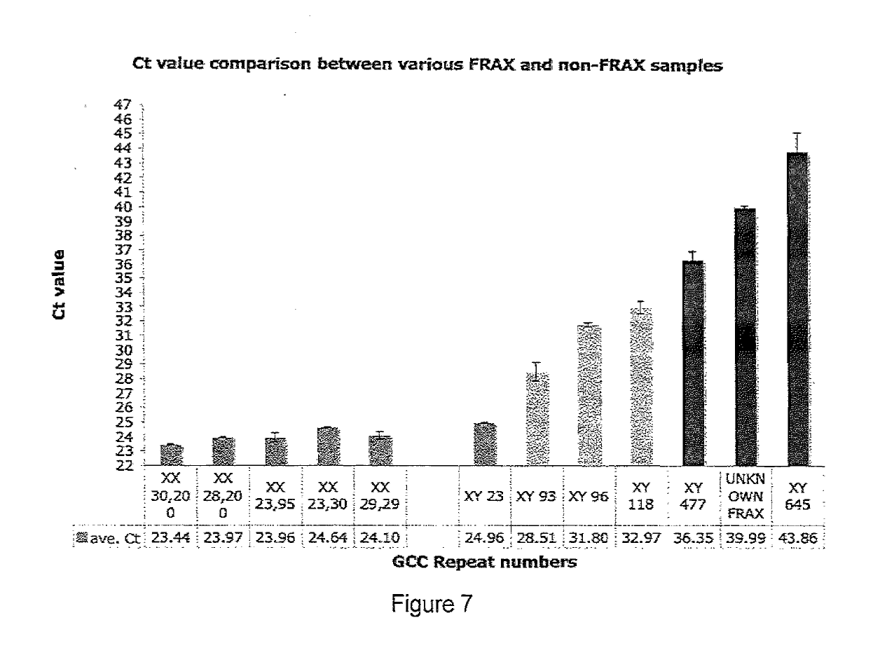

The Ct values for normal, premutation males (93, 96, 118 repeats), and full mutation FRAX males samples (477, Unknown of >200 repeats, 645 repeats) were statistically different from each other (p-value<0.05, ANOVA). When all FRAX samples were compared to non-FRAX samples, a lower Ct value was observed in all non-FRAX samples (p<0.05). FIG. 7 show the actual Ct values observed. These data show that the CGG repeat length in FRAX DNA can be predicted and both premutation and full FRAX mutation in males can be screened based on the Ct value.

Consistent range of Ct values (Ct) can be detected quantitatively in the SSMDA product based on the number of CGG-repeats using various FRAX full mutations, premutation, and non-FRAX gDNA in two independent experiments. These experiments demonstrate that this novel approach is effective in detecting the differences in CGG repeat length to discriminate between FRAX full mutation, premutation, and non-FRAX individuals as well as estimate the CGG repeat copy number.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed:

1. A method of diagnosing a human subject afflicted with fragile X syndrome, wherein said fragile X syndrome is the result of an expansion of the CGG triplet repeat region of the FMR1 gene, said method comprising:
a) obtaining a sample of genomic DNA from said subject;
b) contacting said sample with about 5-10 pairs of nested primers flanking the CGG triplet repeat region of said FMR1 gene;
c) amplifying said CGG triplet repeat region of said FMR1 gene using Phi29 DNA polymerase for site specific multiple displacement amplification (SSMDA);
d) quantifying the number of said CGG triplet repeats present in said CGG triplet repeat region of said FMR1 gene using either real-time PCR or real-time SSMDA, wherein if the number of CGG triplet repeats in the CGG triplet repeat region is more than about 200 CGG repeats, then said subject has fragile X syndrome.

2. The method of claim 1, wherein said sample of genomic DNA is contacted with at least 2 primers selected from the group consisting of SEQ ID NO, 1-19.

3. A method of diagnosing a human subject with a fragile X syndrome premutation, wherein said fragile X syndrome is the result of an expansion of the CGG triplet repeat region of the FMR1 gene, wherein said subject is not afflicted with fragile X syndrome but is at-risk of having progeny with fragile X syndrome, said method comprising:
a) obtaining a sample of genomic DNA from said subject;
b) contacting said sample with about 5-10 pairs of nested primers flanking the CGG triplet repeat region of said FMR1 gene;
c) amplifying said CGG triplet repeat region of said FMR1 gene using Phi29 DNA polymerase for site specific multiple displacement amplification (SSMDA);
d) quantifying the number of said CGG triplet repeats present in said CGG triplet repeat region of said FMR1 using either real-time PCR or real-time SSMDA, wherein if the number of CGG triplet repeats in the CGG triplet repeat region is from about 60 to about 200 CGG repeats, then said subject has a fragile X premutation and is at-risk of having progeny with fragile X syndrome.

4. A method of diagnosing a human subject with a fragile X syndrome intermediate premutation, wherein said fragile X syndrome intermediate premutation is the result of an expansion of the CGG triplet repeat region of the FMR1 gene, wherein said subject is not afflicted by fragile X syndrome but is at-risk of having progeny with fragile X syndrome, said method comprising:
a) obtaining a sample of genomic DNA from said subject;
b) contacting said sample with about 5-10 pairs of nested primers flanking the CGG triplet repeat region of said FMR1 gene;
c) amplifying said CGG triplet repeat region of said FMR1 gene using Phi29 DNA polymerase for site specific multiple displacement amplification (SSMDA);
d) quantifying the number of said CGG triplet repeats present in said CGG triplet repeat region of said FMR1 using either real-time PCR or real-time SSMDA, wherein if the number of CGG triplet repeats in the CGG triplet repeat region is from about 45 to about 60 CGG repeats, then said subject has an intermediate fragile X premutation and is at-risk of having progeny with fragile X syndrome.

5. A method of diagnosing a human subject afflicted with fragile X syndrome, wherein said fragile X syndrome is the result of an expansion of the CGG triplet repeat region of the FMR1 gene, said method comprising:
a) obtaining a sample of genomic DNA from said subject;
b) digesting said genomic DNA with at least one restriction enzyme wherein said restriction enzyme excises a region of genomic DNA comprising said CGG triplet repeat region of the FMR1 gene;
c) ligating said digested DNA to form circularized DNA comprising said CGG triplet repeat region of the FMR1 gene;
d) contacting said circularized DNA with about 5-10 pairs of nested primers flanking the CGG triplet repeat region of said FMR1 gene;
e) amplifying said CGG triplet repeat region of said FMR1 gene using Phi29 DNA polymerase for site specific multiple displacement amplification (SSMDA);
f) quantifying the number of CGG triplet repeats present in said CGG triplet repeat region of said FMR1 gene using either real-time PCR or real-time SSMDA, wherein if the number of CGG triplet repeats in the CGG triplet repeat region is more than about 200 CGG repeats, then said subject has fragile X syndrome.

6. A method of diagnosing a human subject with a fragile X premutation, wherein said fragile X premutation is the result of an expansion of the CGG triplet repeat region of the FMR1 gene, wherein said subject is not afflicted with fragile X syndrome but is at-risk of having progeny with fragile X syndrome, said method comprising:
a) obtaining a sample of genomic DNA from said subject;
b) digesting said genomic DNA with at least one restriction enzyme wherein said restriction enzyme excises a region of genomic DNA comprising said CGG triplet repeat region of the FMR1 gene;
c) ligating said digested DNA to form circularized DNA comprising said CGG triplet repeat region of the FMR1 gene;
d) contacting said circularized DNA with about 5-10 pairs of nested primers flanking the CGG triplet repeat region of said FMR1 gene;
e) amplifying said CGG triplet repeat region of said FMR1 gene using Phi29 DNA polymerase for site specific multiple displacement amplification (SSMDA);
f) quantifying the number of said CGG triplet repeats present in said CGG triplet repeat region of said FMR1 gene using either real-time PCR or real-time SSMDA, wherein if the number of CGG triplet repeats in the CGG triplet repeat region is from about 60 to about 200 CGG repeats, then said subject has a fragile X premutation and is at-risk of having progeny with fragile X syndrome.

7. A method of diagnosing a human subject with an intermediate fragile X premutation, wherein said fragile X intermediate premutation is the result of an expansion of the CGG triplet repeat region of the FMR1 gene, wherein said subject is not afflicted with fragile X syndrome but is at-risk of having progeny with fragile X syndrome, said method comprising:
a) obtaining a sample of genomic DNA from said subject;
b) digesting said genomic DNA with at least one restriction enzyme wherein said restriction enzyme excises a region of genomic DNA comprising said CGG triplet repeat region of the FMR1 gene;
c) ligating said digested DNA to form circularized DNA comprising said CGG triplet repeat region of said FMR1 gene;
d) contacting said circularized DNA with about 5-10 pairs of nested primers flanking the CGG triplet repeat region of said FMR1 gene;
e) amplifying said CGG triplet repeat region of said FMR1 gene using Phi29 DNA polymerase for site specific multiple displacement amplification (SSMDA);
f) quantifying the number of said CGG triplet repeats present in said CGG triplet repeat region of said FMR1 gene using either real-time PCR or real-time SSMDA, wherein if the number of CGG triplet repeats in the CGG triplet repeat region is from about 45 to about 60 CGG repeats, then said subject has an fragile X intermediate premutation and is at-risk of having progeny with fragile X syndrome.


SEQUENCE LISTING (25 sequences)

SEQ ID NO: 1 - DNA, 1050 bp, Artificial Sequence
  chemically synthesized
cagacaccccctcccgcggaatcccagagaggccgaactgggataaccggatgcatttgatttcccacgccactgagtgcacctctgcagaaatgggcgttctggccctcgcgaggcagtgcgacctgtcaccgcccttcagccttcccgccctccaccaagcccgcgcacgcccggcccgcgcgtctgtctttcgacccggcaccccggccggttcccagcagcgcgcatgcgcgcgctcccaggccacttgaagagagagggcggggccgaggggctgagcccgcggggggagggaacagcgttgatcacgtgacgtggtttcagtgtttacacccgcagcgggccgggggttcggcctcagtcaggcgctcagctccgtttcggtttcacttccggtggagggccgcctctgagcgggcggcgggccgacggcgagcgcgggcggcggcggtgacggaggcgccgctgccagggggcgtgcggcagcgcggcggcggcggcggcggcggcggcggcggaggcggcggcggcggcggcggcggcggcggctgggcctcgagcgcccgcagcccacctctcgggggcgggctcccggcgctagcagggctgaagagaagatggaggagctggtggtggaagtgcggggctccaatggcgctttctacaaggtacttggctctagggcaggccccatcttcgcccttccttccctcccttttcttcttggtgtcggcgggaggcaggcccggggccctcttcccgagcaccgcgcctgggtgccagggcacgctcggcgggatgttgttgggagggaaggactggacttggggcctgttggaagcccctctccgactccgagaggccctagcgcctatcgaaatgagagaccagcgaggagagggttctctttcggcgccgagccccgccggggtgagctggggatgggcgagggccggcggcaggtactagagccgggcgggaagggccgaaatcggcgctaagtgacggcgatggcttattccccctttcctaaacatcatctcccag

SEQ ID NO: 2 - DNA, 20 bp, Artificial Sequence
  chemically synthesized
aactgggataaccggatgca

SEQ ID NO: 3 - DNA, 19 bp, Artificial Sequence
  chemically synthesized
agtgcacctctgcagaaat

SEQ ID NO: 4 - DNA, 19 bp, Artificial Sequence
  chemically synthesized
aggcagtgcgacctgtcac

SEQ ID NO: 5 - DNA, 17 bp, Artificial Sequence
  chemically synthesized
ttcccgccctccaccaa

SEQ ID NO: 6 - DNA, 20 bp, Artificial Sequence
  chemically synthesized
accccggccggttcccagca

SEQ ID NO: 7 - DNA, 18 bp, Artificial Sequence
  chemically synthesized
aggccacttgaagagaga

SEQ ID NO: 8 - DNA, 19 bp, Artificial Sequence
  chemically synthesized
agcgttgatcacgtgacgt

SEQ ID NO: 9 - DNA, 17 bp, Artificial Sequence
  chemically synthesized
cagcgggccgggggttc

SEQ ID NO: 10 - DNA, 18 bp, Artificial Sequence
  chemically synthesized
tcacttagcgccgatttc

SEQ ID NO: 11 - DNA, 19 bp, Artificial Sequence
  chemically synthesized
cccatccccagctcacccc

SEQ ID NO: 12 - DNA, 19 bp, Artificial Sequence
  chemically synthesized
accctctcctcgctggtct

SEQ ID NO: 13 - DNA, 18 bp, Artificial Sequence
  chemically synthesized
gcctctcggagtcggaga

SEQ ID NO: 14 - DNA, 20 bp, Artificial Sequence
  chemically synthesized
cagtccttccctcccaacaa

SEQ ID NO: 15 - DNA, 17 bp, Artificial Sequence
  chemically synthesized
tggcacccaggcgcggt

SEQ ID NO: 16 - DNA, 18 bp, Artificial Sequence
  chemically synthesized
cctgcctcccgccgacac

SEQ ID NO: 17 - DNA, 18 bp, Artificial Sequence
  chemically synthesized
ggaaggaagggcgaagat

SEQ ID NO: 18 - DNA, 21 bp, Artificial Sequence
  chemically synthesized
gacggaggcgccgctgccagg

SEQ ID NO: 19 - DNA, 20 bp, Artificial Sequence
  chemically synthesized
tgggctgcgggcgctcgagg

SEQ ID NO: 20 - DNA, 15 bp, Artificial Sequence
  chemically synthesized
cgccgccgccgccgc

SEQ ID NO: 21 - DNA, 15 bp, Artificial Sequence
  chemically synthesized
cgccgccgccgccgc

SEQ ID NO: 22 - DNA, 17 bp, Artificial Sequence
  chemically synthesized
cagcgggccgggggttc

SEQ ID NO: 23 - DNA, 20 bp, Artificial Sequence
  chemically synthesized
cctggcagcggcgcctccgt

SEQ ID NO: 24 - DNA, 19 bp, Artificial Sequence
  chemically synthesized
gaagtgaaaccgaaacgga

SEQ ID NO: 25 - DNA, 19 bp, Artificial Sequence
  chemically synthesized
gaagtgaaaccgaaacgga